(19) United States
(12) Patent Application Publication
(10) Pub. No.: US 2008/0268483 A1
(43) Pub. Date: Oct. 30, 2008

(54) Methods for a Global Assay of Coagulation and Fibrinolysis

(75) Inventors: Neil A. Goldenberg, Aurora, CO (US); Marilyn J. Manco-Johnson, Greenwood Village, CO (US); Linda Jacobson, Denver, CO (US); William E. Hathaway, Denver, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: The Regents of the University of Colorado

(21) Appl. No.: 11/575,853

(22) PCT Filed: Sep. 22, 2005
(86) PCT No.: PCT/US05/33999

§ 371 Date: Feb. 11, 2008

(30) Foreign Application Priority Data
Sep. 22, 2004 (US) 60/612580

(51) Int. Cl. C12Q 1/56

(52) U.S. Cl. 435/13

(57) ABSTRACT

The present disclosure concerns methods of analyzing both clot formation and fibrinolysis in a sample, preferably simultaneously. In certain embodiments, the methods may comprise adding a small amount of at least one activator of coagulation and at least one activator of fibrinolysis to a sample and analyzing the sample for kinetic parameters related to clot formation and fibrinolysis. In another embodiment, the methods may comprise analyzing a sample from a subject for clot formation and fibrinolysis and detecting or diagnosing a disease or condition and/or applying information obtained from analyzing clot formation and fibrinolysis to determine a treatment for a medical condition of the subject.

### RELATED APPLICATIONS

The present application claims the benefit under 35 U.S.C. §119(e) of provisional U.S. patent application Ser. No. 60/612,580, filed on Sep. 22, 2004.

### FIELD

The present invention relates to methods for combined assessment of coagulation (clot formation) and fibrinolytic capacity (clot lysis) in a sample, such as whole blood, plasma, platelet rich plasma and/or platelet-poor plasma. In preferred embodiments, coagulation and clot lysis are measured simultaneously. In various embodiments, parameters of clotting and/or fibrinolysis derived from the disclosed methods may be used for the detection, diagnosis and/or prognosis of various disease states that affect hemostatic balance, such as hemophilia, von Willebrand's disease and other bleeding or prothrombotic conditions. The disclosed methods are of use to assess an individual's prothrombotic and/or hemorrhagic tendencies in a wide variety of conditions, such as trauma, acute coronary events/syndromes, cardiac bypass, organ transplantation, intensive care, diagnostic surgical biopsies, or other surgical or medical procedures.

### BACKGROUND

Predicting and preventing catastrophic bleeding or excessive clotting (“thrombotic”) episodes in patients with coagulation disorders remains a critical, and largely unrealized, medical challenge. Unlike individual molecular tests, assays that evaluate net clotting potential or the generation of (a) key enzymatic player(s) in the clotting system offer the potential to assist in the prediction of individual bleeding and thrombotic risk at a given point in time, and even the possibility to tailor a specific preventive medical approach to a particular patient based upon the net balance of his/her clotting system. Historically, such “global assays” have rarely been practical for clinical application. Over the past few years, technological advances have made the prospect of a clinically useful global assay more tenable. Yet, to date very few such global assays have been designed to evaluate both the clot formation (“coagulation”) and clot breakdown (“fibrinolysis”) abilities of the blood, each of which is an important component of the coagulation system. Defects in each of these functions have been found, for example, in severe hemophiliacs, as well as in a variety of bleeding and thrombotic disorders.

Despite many scientific advances in recent years to better understand bleeding and thrombotic disorders on the level of gene mutations, such diseases continue to cause long-term disability in a significant subset of patients. The ability to predict catastrophic bleeding or clotting episodes is an important goal for patients and their treating clinicians in order to maximize the potential for an enduring high level of patient functioning. This goal has remained largely elusive because individual molecular markers of coagulation do not provide an overall picture of an individual's hemostatic balance at a given time.

The present emphasis on further elucidating the molecular basis of coagulation diseases, while essential to the development of more targeted therapeutic approaches, has to date inadequately addressed many important questions that continue to complicate patient care on a daily basis. Clinicians are still unable, for example, to distinguish among hemophilia B patients with similar factor IX levels those patients who are at greatest risk for clinically-significant bleeding and who may therefore benefit from aggressive prophylactic or therapeutic interventions. Similarly, despite much progress on the molecular level in the field of thrombophilia research, most recently with the identification of the Factor V Leiden and prothrombin 20210 mutations, many patients with thrombosis have no detectable thrombophilia trait. Even more numerous are patients who have one or more identifiable thrombophilia traits. For these patients, there is as yet little medical understanding of composite prothrombotic risk upon which to guide management decisions regarding thromboprophylaxis and antithrombotic therapy. Since the understanding of bleeding and thrombotic disorders has become increasingly molecular, the number of identifiable disorders of hemostasis has expanded and at the same time, the gap in understanding between the molecular etiologies of these varied disorders and their net impact on the clotting system continues to widen. Among patients with similar molecular defects, there is often considerable variation in clinical phenotype, leading to much difficulty with regard to patient care. Scientists and clinicians in the field of coagulation research have recently recognized the serious need for a global assessment of hemostasis to help distill the effects of complex or multiple defects, to streamline a presently extensive and expensive panel of diagnostic coagulation and fibrinolytic assays, and to better tailor management guidelines and recommendations regarding prophylaxis and treatment to individual patients.

Unlike a panel of individual molecular tests, assays that evaluate net clotting potential, or the generation of key enzymes in the coagulation system (e.g., thrombin), provide a more complete fingerprint of a patient's clotting state. At various timepoints in the history of modern coagulation research, such global tests have been developed, but their clinical utility has most often been impeded by concerns of physiologic relevance, reproducibility, complexity, cost, timely results, and the requirement for continuous or multiple blood sampling.

Among the classical global assays, only the thromboelastogram (TEG) and euglobulin lysis time (ELT) assay continue to be used clinically. A recent rise of interest in global tests of coagulation and fibrinolysis has brought attention to the need for global assays sensitive to an array of hemostatic alterations. Using zymogen forms of procoagulants and anticoagulants at their mean physiologic concentrations in plasma, to which TF (tissue factor) and calcium were added, the generation of thrombin has been measured and enhanced thrombin generation has been demonstrated in states of prothrombin excess and antithrombin deficiency (Butenas et al, 1999). The need for serial subsampling of plasma has been avoided by utilizing a minimally-consumed chromogenic (more recently, a fluorogenic) thrombin-specific substrate, which permitted continuous registration of thrombin generation in plasma (Hemker and Beguin, 1995, 2000; Hemker et al., 2000). This technology has become increasingly applied in clinical coagulation research in the past few years (Turacek et al., 2003; Quiroga et al., 2003; Giansily-Blaizot et al., 2003; Faber et al., 2003). This assay has also been used in a modified format to contribute to the understanding of coagulation in newborn infants (Cvirn et al., 1999, 2003).

However, thrombin generation assays, while providing an important representation of coagulability, do not assess the fibrinolytic activities, a component of hemostasis with important clinical relevance. Altered fibrinolysis has been demonstrated not only in the physiologic states of pregnancy and the neonatal period, but also has been implicated in numerous bleeding and prothrombotic conditions. For example, excessive fibrinolysis is observed in severe hemophilia A (Mosnier et al. 2001) and hepatic cirrhosis (Colucci et al. 2003), and deficient fibrinolysis has been demonstrated in the context of renal failure (Lottermoser et al., 2001) and elevated plasma lipoprotein(a) levels (Palabrica et al. 1995).

Enhanced overall hemostatic potential and reduced fibrinolytic potential in the plasma of pregnant women has been observed using a turbidimetric method involving TF- and thrombin-mediated coagulation activation and tPA (tissue-type plasminogen activator)-enhanced fibrinolysis (He et al., 1999, 2001a). Similar studies have indicated increased overall hemostatic potential in type I diabetic patients (Antovic et al., 2003a), surgically post-menopausal females taking high-dose estrogens (He et al., 2001b), and women with a prior history of pregnancy-associated deep venous thrombosis (Antovic et al., 2003b).

Despite such advances, a need still exists for a global assay that measures both plasma coagulation and fibrinolysis, preferably simultaneously, over a continuous window that is suitable for both pediatric (including neonatal) and adult clinical applications. Such an assay would allow evaluation of an individual's unique net hemostatic balance at any given time and the assessment of prothrombotic and hemorrhagic risk and treatment.

### SUMMARY

The present invention relates to methods and compositions for evaluating clot formation and fibrinolysis in a sample. In one exemplary method designated as Clot Formation and Lysis (CloFAL) assay, a clot is formed in a sample of blood or plasma and thereafter the clot is lysed. The kinetic parameters for formation and lysis of the clot are determined, preferably using a spectrophotometric assay, to assess the individual's net hemostatic balance at a given time, allowing prothrombotic and hemorrhagic risk assessment. In another embodiment, measured parameters can include the maximum amplitude (MA) of spectrophotometric absorbance, the time to maximum turbidity (T₁), the time to completion of the first phase of decline in turbidity (T₂), and the area under the curve (AUC) over measured time intervals. From such measurements, the coagulation index (CI) and fibrinolytic index (FI) may be determined. CI, FI and/or individual CloFAL parameters are of use to detect or diagnose prothrombotic and/or hemorrhagic diseases or conditions and to develop therapeutic treatments tailored to the individual's net hemostatic balance.

In certain embodiments, involving continuous measurement of clot lysis and clot formation in a sample, the information obtained is more comprehensive and more directly related to actual physiological conditions for clot formation and lysis in the body than presently available assays. The disclosed methods and compositions allow the rapid and inexpensive assessment of the hemostatic balance in an individual over time.

In one embodiment, clot formation and fibrinolysis may be performed in a container or test cell, including but not limited to 96-well microtiter plates, into which a sample (e.g. fresh or freeze-thawed, platelet-poor plasma) and appropriate reagents have been added. An exemplary apparatus of use may include a sample, one or more reagents, buffer, a reagent chamber, and a detection instrument, such as a spectrophotometer. In more particular embodiments, the reagents added to the reagent chamber may include small amounts of tissue factor (TF) and/or tissue-type plasminogen activator (tPA). Where exemplary containers exhibit multiple sample compartments, such as a 96-well plate, the sample may preferably be analyzed in replicates, such as duplicate or triplicate wells of a 96-well plate. An advantage of the disclosed methods is that the amount of sample required to assay may be relatively small, for example 75 μL of plasma sample per well.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 shows an example of a CloFAL curve from standard normal pooled adult platelet-poor plasma, demonstrating principal CloFAL parameters.

FIG. 2 shows an example of a CloFAL curve from a normal healthy adult, a newborn infant, a normal child and a pregnant woman. (SNP=standard normal pooled adult plasma).

FIGS. 3A and 3B show an example of scatterplots of (3A) coagulation index (CI) and (3B) fibrinolytic index (FI) values by subject group. Group medians are indicated by horizontal bars.

FIGS. 4A and 4B show an example of the influence of plasma (4A) fibrinogen concentration and (4B) factor VIII activity upon the CloFAL curve.

FIG. 5 shows an example of CloFAL curves for selected procoagulant factor deficiency states (e.g. factors II, V, IX, and X). A vertical line is indicated at 30 minutes, given that the cumulative AUC at 30 minutes is one important parameter of coagulation index CI.

FIG. 6 shows an example of CloFAL curves for selected fibrinolytic alterations for PAI-1 (plasminogen activator inhibitor-1) deficiency, Amicar (aminocaproic acid) treatment and inhibition of TAFI (thrombin activatable fibrinolytic inhibitor) activation by PTCI (potato tuber carboxypeptidase inhibitor). The PAI-1 deficient sample was obtained 24 hours following a therapeutic dose of aminocaproic acid.

FIG. 7 represents some effects of heparin treatment and its reversal upon the CloFAL curve.

FIGS. 8A and 8B show an example of hemostatic response to therapeutic or prophylactic recombinant human FVIII administration in severe hemophilia A, as measured by the CloFAL global assay. FIG. 8A represents a baseline CloFAL curve following a treatment in an adult patient with severe hemophilia A during a bleeding episode. FIG. 8B represents a baseline CloFAL curve following a treatment in a child with severe hemophilia A.

Table 1A shows exemplary median CloFAL CI and correlative laboratory test values (with interquartile ranges) in healthy term infants, children, adults, and pregnant women at term.

Table 1B shows exemplary median CloFAL FI and correlative laboratory test values (with interquartile ranges) in healthy term infants, children, adults, and pregnant women at term.

Table 2 represents a CloFAL assay with CI values from individual coagulation factor-deficient patient plasmas.

Table 3 represents distributions of age and laboratory and clinical disease severity among children and adults with or without factor VIII deficiency.

Table 4 shows exemplary median laboratory values for the CloFAL global assay, aPTT, one-stage FVIII assay, and vWF Ag ELISA among children and adults with or without factor VIII deficiency.

Table 5 represents sensitivities of the CloFAL global assay and aPTT for different laboratory severities of factor VIII deficiency.

### DESCRIPTION OF ILLUSTRATIVE EMBODIMENTS

### Definitions

Terms that are not otherwise defined herein are used in accordance with their plain and ordinary meaning.

As used herein, “a” or “an” may mean one or more than one of an item.

As used herein, “modulation” refers to a change in the level or magnitude of an activity or process. The change may be either an increase or a decrease. For example, modulation may refer to either an increase or a decrease in activity or levels. Modulation may be assayed by determining any parameter that indirectly or directly affects or reflects coagulation or fibrinolysis or the combination of coagulation and fibrinolysis.

In the following section, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description.

### General Considerations for Clotting and Fibrinolysis Assays

The coagulation and fibrinolysis systems are extraordinarily complex and interwoven processes that involve dozens of proteins, each of which may become dysfunctional or deficient due to genetic variation or mutation, traumatic injury and/or a disease state. Traditionally used coagulation assays include tests like aPTT (activated partial thromboplastin time) that focus on binary events, which do not disclose the events occurring at the molecular level. For optimal care of patients, understanding the positive and negative dynamics of clotting is important to prescribe the proper treatment for the individual.

Healthcare providers are in need of an inexpensive and easily administered global hemostatic assay. Because of the nature of hemostasis as a dynamic on-going process, a method that can track clot formation and lysis over time would be extremely beneficial from a clinical perspective. The application of such methods is important for patients with hemostatic disorders, trauma patients and those undergoing any type of surgical treatment such as invasive techniques that frequently involve bleeding and/or clot formation. Other situations where these techniques would be extremely useful include cardiovascular interventions, organ transplantation and many intensive care situations.

Methods and compositions of a global assay to analyze both the formation and dissolution of a clot are disclosed herein. An inexpensive and reliable global assay assessing both systems will promote optimal application of a physician's resources to diagnose particular blood factor deficiencies and other conditions of altered hemostasis, monitor response to drug regimen and enhance treatment efficiency, leading to a decreased loss of function, decreased health care cost and decreased loss of life.

In vivo, clot formation and subsequent clot lysis do not ordinarily occur in a normal individual absent physiological causes, such as physical trauma to blood vessels, pathological blood disorders or therapeutically induced blood reactions. Similarly, under in vitro conditions, clot formation and clot lysis reactions may be absent or retarded if the medium or environment into which the blood sample is collected retards those reactions. Clot formation and clot lysis reactions may be controlled in vivo by the presence of therapeutically administered reagents. In order to accomplish in vitro measurement of blood clot formation and clot lysis, traces of additional reagents may be added to the blood sample to induce or maximize clot formation and clot lysis in the mixture. These reagents may include small amounts of TF (tissue factor) and/or tPA (tissue-type plasminogen activator) or other known activators of clot formation and/or lysis.

Typically, “global” coagulation and fibrinolysis assays are more efficient at detecting specific types of coagulation deficiency. Assays that incorporate the effect of blood cells are more holistically inclusive of hemostatic dynamics, but given turbidity and other technical limitations are not readily amenable to inexpensive and rapid spectrophotometric or other analyses. Current methods focus on measuring coagulation and/or fibrinolysis during a particular snapshot of time, instead of tracking the complete process over the duration of the event, from clotting cascade initiation to final fibrinolysis.

### The CloFAL Assay

Advantages of the CloFAL (Clot Formation and Lysis) assay include reliable results that correlate with aPTT and PT (prothrombin time) assays, using inexpensive and readily available reagents. Because the assay utilizes turbimetric monitoring instead of fluorometric or luminescent tagged reagents, the cost and availability are improved. The equipment used to monitor clot formation and lysis, for example a spectrophotometer, is simple, easy to use, and readily available in most research and clinical laboratories and does not require any extensive training of the operator. The turbidometric assay is straightforward since external activators such as additional thrombin are not added to the assay mix. As thrombin may function as a rate-limiting enzyme in hemostasis in vivo, avoiding the addition of thrombin simplifies interpretation of the assay results and may increase sensitivity for coagulopathy. The assay is very sensitive and requires a short time period, typically in the time range of three hours or less. Since the CloFAL assay measures the process from cascade initiation through clot lysis, it provides more complete data than presently used methods.

The CloFAL assay typically manifests two phases, rather than a single phase, of decline in turbidity associated with fibrinolysis. The evaluation of FI in the context of changes in the duration of the first phase of decline in turbidity with modulations in known key components of the fibrinolytic system has suggested that the CloFAL assay is sensitive to altered states of fibrinolysis, including those induced by exercise, PAI-1 deficiency, aminocaproic acid and inhibition of TAFI (thrombin activatable fibrinolytic inhibitor) activation.

### Disadvantages of Present Assay Systems

One present assay system, thromboelastography, uses whole blood and is available at point of care (POC) facilities, but it focuses on the mechanical characteristics of clot formation and fibrinolysis and not physiological conditions. In addition, this technology is limited by the requirement for a fresh blood specimen. Surface Plasmon Resonance (SPR) senses surface interactions and Free Oscillation Rheometry (FOR) senses interactions within material but these assays are developmentally in their infancy and demand highly specialized equipment and reagents, along with skilled operators. Clot Signature Analyzer (CSA) uses non-anticoagulated whole blood to measure clot formation. Calibrated Automated Thrombogram (CAT) measures up to 100 samples/hour, both hypo- and hyper-coagulation states, is relatively sensitive to inherited antithrombin (AT) deficiency and is sensitive in platelet-poor plasma (PPP). However, it has lower sensitivity to protein anticoagulant systems and low responsiveness with platelet rich plasma (PRP), particularly to various disease or drug treatment states. ProC Global (PCG) assay is sensitive to protein C and useful as a screening test for protein C, protein S, activated protein C resistance (APCR), and lupus anticoagulant coagulopathies, but has lower AT sensitivity. However, none of these methods is designed to assess fibrinolysis.

The disadvantages that each of these assays presents compared to assessing both the formation and dissolution of a clot as presented herein are the lack of complete assessment of a sample over time and the ease of use of the measuring instrument. Both components of the process are important in understanding the entire physiological process of clot formation and fibrinolysis in order to accurately diagnose and treat conditions associated with these systems.

### Uses of CloFAL Assay

### Evaluating and Monitoring Fibrinolytic Capacity

Whether or not cell destruction can be minimized after physiological events such as myocardial infarctions, stroke or gangrene may depend, in part, upon the existence of pathological or therapeutically induced fibrinolysis. In order to eliminate or minimize such cell destruction in an individual who has undergone or is undergoing a stroke, heart attack or similar event, it would be useful to rapidly ascertain whether the individual's clot lysis ability is within a normal range of lytic response times. By comparing the individual's specific lytic response time to an average lytic response time of a normal, non-pathogenic individual, or within a given individual over time, a treating physician may determine whether the patient's specific lytic response capability needs to be treated or otherwise taken into consideration.

Under conditions when arterial or venous thrombosis has occurred or is likely to occur, such as during and after surgery, it becomes critical that the treating physician has reliable information available about an individual's fibrinolytic processes. For example, clot formation is especially likely to occur during cardiac surgery utilizing extra-corporeal passage of blood. Although clotting during cardiac surgery may be minimized through use of heparin or other anticoagulants, a surgical patient's natural lytic ability can help avoid surgical complications by dissolving any clots that form. If a particular surgical patient's lytic ability is impaired, a physician may elect to administer thrombolytic agents to maintain a particular level of lytic activity and to avoid the possibility of permanent and disabling clot formation occurring during surgery. To maintain a desired level of lytic activity, it would be useful to assess whether the administration of a thrombolytic agent had the desired effect upon the surgical patient.

Furthermore, when a deep venous thrombosis or pulmonary embolism is veno-occlusive and/or extensive, compromising venous or pulmonary function or risking chronic venous insufficiency due to venous valvular damage, thrombolytic therapy may be indicated. Such therapy would be better monitored (and its bleeding complications potentially minimized) through use of an assay designed to measure fibrinolytic capacity of plasma at a given time or within a selected time period, such as pre-treatment, during treatment, or post-treatment.

### Evaluating and Monitoring Coagulation Potential

In the setting of bleeding disorders and known coagulation factor deficiencies, measurement of the individual patient's coagulation potential would be of use in order to tailor dose intensity and duration of therapies and/or prophylactic measures (e.g., the administration of factor concentrates or recombinant proteins) to the type and severity of hypocoagulability exhibited by the patient's plasma at the time of the assessment and intervention. Similarly, in the context of prothrombotic conditions, measurement of the individual patient's coagulative capacity would be of use in order to tailor dose intensity and duration of antithrombotic therapies and/or prophylactic measures (e.g., the administration of anticoagulants or thrombin inhibitors) to the type and severity of the patient's hypercoagulable state.

### Clotting Process

It is essential for survival to control the flow of blood following vascular injury. The process of blood clotting and the subsequent dissolution of the clot, following repair of the injured tissue, is termed hemostasis. The process of hemostasis is composed of four principle events that occur sequentially following the loss of vascular integrity. The first phase includes vascular constriction that limits the flow of blood to the area of injury. Tissue factor (also known as tissue thromboplastin) is exposed on the injured vascular endothelium, initiating the coagulation cascade, producing thrombin, which acts on fibrinogen to form fibrin. Thrombin also activates the platelets that have adhered to the injured endothelium, which then aggregate, forming a temporary, loose platelet plug. Further platelet clumping is mediated by fibrinogen, as well as by exposed collagen on the injured endothelium. Activated platelets release adenosine-5′-diphosphate (ADP) as well as various proteins that in turn activate additional regulators, such as serotonin, phospholipids, lipoproteins, and other proteins that modulate the coagulation cascade. As the coagulation cascade ensues, the platelet plug is stabilized by a fibrin mesh, forming an organized thrombus, or clot.

For resumption of normal blood flow to occur following tissue repair the clot must be dissolved. This occurs through the action of plasmin, which cleaves fibrin, and thereby disorganizes the clot. Plasmin is regulated by activators and inhibitors of its enzymatic pathways, as further discussed below.

Platelet Activation and von Willebrand Factor (vWF)

In order for hemostasis to occur, platelets must adhere to exposed collagen, release the contents of their granules, and aggregate. The adhesion of platelets to the collagen exposed on endothelial cell surfaces is mediated by von Willebrand factor. The function of vWF is to act as a bridge between a specific glycoprotein on the surface of platelets and collagen fibrils. vWF binds to and stabilizes coagulation factor VIII. Binding of factor VIII by vWF is required for normal survival of factor VIII in the circulation. von Willebrand factor is a complex multimeric glycoprotein that is produced by and stored in the α-granules of platelets. It is also synthesized by megakaryocytes and is found associated with subendothelial connective tissue.

As indicated above, the initial activation of platelets is induced by thrombin binding to specific receptors on the surface of platelets, thereby initiating a signal transduction cascade. The thrombin receptor is coupled to a G-protein that, in turn, activates phospholipase C (PLC). Then PLC hydrolyzes phosphatidylinositol-4, 5 bisphosphate (PIP₂) contributing to the formation of inositol triphophate (IP₃) and diacylglycerol (DAG). As a result IP₃ induces the release of intracellular Ca²⁺ stores, and DAG activates protein kinase C (PKC).

Intracellular Ca²⁺ and collagen to which the platelets adhere lead to the activation of phospholipase A₂ (PLA₂), which then hydrolyzes membrane phospholipids to release arachidonic acid. The arachidonic acid release causes an increase in the production and subsequent release of thromboxane A₂ (TXA₂). Myosin light chain kinase (MLCK) is another enzyme activated by the released intracellular Ca²⁺. This results in an altered platelet morphology and motility via a phosphorylation event.

A 47 kDa protein is phosphorylated by PKC which in turn induces release of platelet granule contents such as ADP, further stimulating platelets and increasing the overall activation cascade. This results in the modification of the platelet membrane, allowing fibrinogen to adhere to two platelet surface glycoproteins and results in fibrinogen-induced platelet aggregation. Activation of platelets is required for their consequent aggregation to a platelet plug. An equally significant role of activated platelet surface phospholipids is the activation of the coagulation cascade.

### Factors Involved in Clotting

| Factor | Common Name(s) | Pathway |
|---|---|---|
| Prekallikrein | Fletcher factor | Intrinsic |
| High molecular | contact activation cofactor; | Intrinsic |
| weight kininogen | Fitzgerald, Flaujeac Williams factor | |
| (HMWK) | | |
| I | Fibrinogen | Both |
| II | Prothrombin | Both |
| III | Tissue Factor | Extrinsic |
| IV | Calcium | Both |
| V | Proaccelerin, labile factor, | Both |
| | accelerator (Ac-) globulin | |
| VI (Va) | Accelerin | |
| VII | Proconvertin, serum prothrombin | Extrinsic |
| | conversion accelerator (SPCA), | |
| | cothromboplastin | |
| VIII | Antihemophiliac factor A, | Intrinsic |
| | antihemophilic globulin (AHG) | |
| IX | Christmas Factor, | Intrinsic |
| | antihemophilic factor B, plasma | |
| | thromboplastin component (PTC) | |
| X | Stuart-Prower Factor | Both |
| XI | Plasma thromboplastin antecedent | Intrinsic |
| | (PTA) | |
| XII | Hageman Factor | Intrinsic |
| XIII | Protransglutaminase, | Both |
| | fibrin stabilizing factor (FSF), | |
| | fibrinoligase | |

### The Clotting Cascades

The intrinsic cascade is initiated when contact is made between blood and exposed endothelial cell surfaces. The extrinsic pathway is initiated upon vascular injury which leads to exposure of tissue factor (TF or factor III), a subendothelial cell-surface glycoprotein that binds phospholipid. The two pathways come together at the activation of factor X to Xa. Factor Xa has a role in the further activation of factor VII to VIIa. Active factor Xa hydrolyzes and activates prothrombin to thrombin. Thrombin can then activate factors XI, VIII and V furthering the cascade. Ultimately the role of thrombin is to convert fibrinogen to fibrin and to activate factor XIII to XIIIa. Factor XIIIa (transglutamase) cross-links fibrin polymers solidifying the clot.

### Intrinsic Clotting Cascade

The intrinsic pathway requires the clotting factors VIII, IX, X, XI, and XII. Also required are the proteins prekallikrein and high-molecular-weight kininogen (HMWK), as well as calcium ions and phospholipids secreted from platelets. Each of these pathway constituents leads to the conversion of factor X to an active factor X, sometimes referred to as factor Xa. Initiation of the intrinsic pathway occurs when prekallikrein, HMWK, factor XI and factor XII are exposed to a negatively charged surface. This is termed the contact phase.

Prekallikrein is converted to kallikrein during the contact phase and in turn activates factor XII to factor XIIa. Factor XIIa can then hydrolyze more prekallikrein to kallikrein, upregulating the response to contact activation of coagulation. Factor XIIa also activates factor XI and leads to the release of bradykinin a potent vasodilator, from high-molecular-weight kininogen.

In the presence of Ca²⁺, factor XIa activates factor IX. Several of the serine proteases of the cascade (II, VII, IX, and X) are gla-containing proenzymes (gla refers to enzymes containing vitamin K-dependent gamma-carboxyglutamate). Activated factor IX (IXa) cleaves factor X at an internal arg-ile bond leading to its activation. Then the tenase complex (Ca²⁺ and factors VIIIa, IXa and X) is formed on the surface of activated platelets. The platelets are activated and then present phosphatidylserine and phosphatidylinositol on their surfaces to form the complex. The role of factor VIII in this process is to act as a receptor, in the form of factor VIIIa, for factors IXa and X. Factor VIIIa is termed a cofactor in the clotting cascade. The activation of factor VIII to VIIIa occurs in the presence of minute quantities of thrombin. As the concentration of thrombin increases, factor VIIIa is ultimately cleaved by thrombin and inactivated. This dual action of thrombin upon factor VIII acts to limit the extent of tenase complex formation and thus the extent of the coagulation cascade.

### Extrinsic Clotting Cascade

The extrinsic pathway is initiated at the site of injury in response to the release of tissue factor (factor III or TF). Tissue factor is a cofactor in the factor VIIa-catalyzed activation of factor X. Factor VIIa, a gla residue containing serine protease, activates factor X by a cleavage event in a manner identical to that of factor IXa of the intrinsic pathway. The activation of factor VII occurs through the action of thrombin or factor Xa. A link between the intrinsic and extrinsic pathways is created by the ability of factor Xa to activate factor VII. An additional link between the two pathways exists through the ability of tissue factor and factor VIIa to activate factor IX The tissue factor—factor VIIa-Ca²-Xa complex is a major site for the inhibition of the extrinsic pathway.

### Activation of Prothrombin to Thrombin

The activation of factor X to factor Xa is the common point in both pathways. Factor Xa activates prothrombin (factor II) to thrombin (factor Ia). Thrombin, in turn, converts fibrinogen to fibrin. The activation of thrombin occurs on the surface of activated platelets. A complex (the prothrombinase complex) is required for this activation that includes platelet phospholipids, phosphatidylinositol and phosphatidylserine, Ca²⁺, factors Va and Xa, and prothrombin. Factor V is a cofactor in the formation of this complex, similar to the role of factor VIII in tenase complex formation. Like factor VIII activation, factor V is activated to factor Va by means of minute amounts of and is inactivated by increased levels of thrombin. Factor Va binds to specific receptors on the surfaces of activated platelets and forms a complex with prothrombin and factor Xa.

Thrombin is a key regulatory enzyme in hemostasis and the inflammatory response. Thrombin binds to and leads to the release of G-protein-coupled protease activated receptors (PARs), specifically PAR-1, -3 and -4. The release of these proteins leads to the activation of numerous signaling cascades that in turn increase release of interleukins such as IL-1 and IL-6, increasing secretion of intercellular adhesion molecule-1 (ICAM-1) and vascular cell adhesion molecule-1 (VCAM-1). The thrombin-induced signaling also leads to increased platelet activation and leukocyte adhesion. Thrombin also stimulates thrombin-activatable fibrinolysis inhibitor (TAFI) thus modulating fibrinolysis (degradation of fibrin clots). TAFI is also known as carboxypeptidase U (CPU) whose activity leads to removal of C-terminal lysines from partially degraded fibrin. This leads to an impairment of plasminogen activation, thereby reducing the rate of fibrin clot dissolution (i.e. inhibiting fibrinolysis).

### Control of Thrombin Levels

The inability of the body to control the circulating level of active thrombin would lead to dire consequences. There are two principal mechanisms by which thrombin activity is regulated. The predominant form of thrombin ill the circulation is the inactive prothrombin, whose activation requires the pathways of proenzyme activation described above for the coagulation cascade. At each step in the cascade, feedback mechanisms regulate the balance between active and inactive enzymes.

The activation of thrombin is also regulated by four specific thrombin inhibitors. Anti-thrombin III is the most important since it can also inhibit the activities of factors IXa, Xa, XIa and XIIa. The activity of antithrombin III is Potentiated via a heparin-mediated conformational change in antithrombin that gives the protein a higher affinity for thrombin as well as its other substrates. This effect of heparin is the basis for its clinical use as an anticoagulant. The naturally occurring heparin activator of antithrombin III is present as heparin and heparan sulfate on the surface of vascular endothelial cells. It is this feature that controls the activation of the intrinsic coagulation cascade. In addition, thrombin activity is also inhibited by other factors, for example heparin cofactor II.

### Activation of Fibrinogen to Fibrin

Fibrinogen (factor I) consists of 3 pairs of polypeptides. The 6 chains are covalently linked near their N-terminals through disulfide bonds. Fibrinopeptide regions of fibrinogen contain several glutamate and aspartate residues, imparting a high negative charge to this region and aiding in the solubility of fibrinogen in plasma. Active thrombin is a serine protease that hydrolyses fibrinogen. Thrombin-mediated release of the fibrinopeptides generates fibrin monomers. These monomers spontaneously aggregate in a regular array, forming a somewhat weak fibrin clot. Thrombin also activates factor XIII, which cross-links fibrin monomers, thereby contracting and stabilizing the clot.

### Fibrinolysis

Fibrinolysis is the process in which blood clots are dissolved. Fibrinolysis is the final step in the natural reparative process that follows clot formation, as when a blood clot which was previously formed in response to blood vessel damage is subsequently dissolved after the damage has been repaired. Fibrinolysis may also be induced or enhanced by the therapeutic administration of thrombolytic agents. Thrombolytic agents are administered to minimize the risks of thrombus progression, pulmonary embolism from a deep venous thrombosis, venous valvular damage that may lead to chronic venous insufficiency, and cellular destruction during myocardial infarction, stroke, or other causes of tissue hypoxia in the setting of arterial thrombosis.

### Dissolution of Fibrin Clots

Degradation of fibrin clots is the function of plasmin, a serine protease that circulates as the inactive proenzyme, plasminogen. As a clot is forming, plasminogen binds to both fibrinogen and fibrin and is incorporated into the clot. Tissue plasminogen activator (tPA) and urokinase are serine proteases that convert plasminogen to plasmin. Inactive tPA is released from vascular endothelial cells following trauma and is activated upon binding to fibrin. Urokinase also exists as a preprotein called prourokinase that is synthesized by epithelial cells in the lining of excretory ducts. Activated tPA cleaves plasminogen to plasmin, which in turn digests fibrin. This results in a soluble degradation product to which neither plasmin nor plasminogen can bind. Following their release, plasminogen and plasmin are rapidly inactivated by their respective inhibitors. The inhibition of tPA activity results from binding to specific inhibitory proteins such as plasminogen activator-inhibitors type 1 (PAI-1) and type 2 (PAI-2).

### Diseases and Conditions

Several bleeding disorders and prothrombotic conditions exist that result from defects in the process of hemostasis. These bleeding conditions have been identified at the level of the proteins of the clotting cascades, platelet activation and function, contact activation and antithrombin function. Perhaps the most widely known inherited bleeding disorder is hemophilia A, or classic hemophilia (a disease referring to the inability to clot blood). It is an X-linked disorder resulting from a deficiency in factor VIII, a key component of the coagulation cascade. There are severe, moderate and mild forms of hemophilia A that reflect the level of active factor VIII in the plasma. Hemophilia B results from deficiencies in factor IX. At least 300 unique factor IX mutations have been identified, 85% are point mutations, 3% are short nucleotide deletions or insertions and 12% are gross gene alterations. Clinical management of hemophilia B is complicated by the fact that, more so than with hemophilia A, the genotype and activity level of factor IX do not necessarily correlate with bleeding phenotype.

Several cardiovascular risk factors are associated with abnormalities in fibrinogen. As a result of the acute-phase response or through other poorly understood mechanisms, elevated plasma fibrinogen levels have been observed in patients with coronary artery disease, diabetes, hypertension, peripheral arterial disease, thrombosis hyperlipoproteinemia and hypertriglyceridemia. In addition, pregnancy, menopause, hypercholesterolemia, use of oral contraceptives and smoking lead to increased plasma fibrinogen levels.

Although rare, there are inherited disorders in fibrinogen. These disorders include afibrinogenemia (a complete lack of fibrinogen), hypofibrinogenemia (reduced levels of fibrinogen) and dysfibrinogenemia (presence of dysfunctional fibrinogen). Afibrinogenemia is characterized by neonatal umbilical cord hemorrhage, ecchymoses, mucosal hemorrhage, internal hemorrhage, and recurrent abortion. The disorder is inherited in an autosomal recessive manner. Hypofibrinogenemia is characterized by fibrinogen levels below 100 mg/dL (normal is 250-350 mg/dL) and can be either acquired or inherited. Symptoms of hypofibrinogenemia are similar to, but less severe than, afibrinogenemia. Dysfibrinogenemias are extremely heterogeneous, affecting any of the functional properties of fibrinogen. Clinical consequences of dysfibrinogenemias include hemorrhage, spontaneous abortion and thromboembolism.

Factor XIII is the proenzyme form of plasma transglutaminase and is activated by thrombin in the presence of calcium ions. Activated factor XIII catalyzes the cross-linking of fibrin monomers. Factor XIII is a tetramer of two different peptides, a and b (forming a₂b₂). Hereditary deficiencies (autosomal recessive) may occur, resulting in the absence of either subunit. Clinical manifestation of factor XIII deficiency is delayed bleeding (although primary hemostasis is normal). Deficiency leads to neonatal umbilical cord bleeding, intracranial hemorrhage and soft tissue hematomas.

Von Willebrand disease (vWD) is due to inherited deficiency in von Willebrand factor (vWF) protein or its function. vWD is the most common inherited bleeding disorder of humans. Using laboratory testing, abnormalities in vWF can be detected in approximately 8000 people per million. Clinically significant vWD occurs in approximately 125 people per million. This is a frequency at least twice that of hemophilia A.

Antithrombin functions to inhibit several activated coagulation factors including thrombin, factor IXa and factor Xa, by forming a stable complex with the various factors. Heparin and heparan sulfates increase the activity of antithrombin at least 1000 fold. Other native anticoagulants include proteins C and S. Clinical manifestations of native anticoagulant deficiency include deep vein thrombosis and pulmonary embolism. Thrombosis may occur spontaneously or in association with surgery, trauma or pregnancy. Treatment of acute episodes of thrombosis is most often by intravenous infusion of unfractionated heparin or subcutaneous administration of low-molecular-weight heparin (for 5-7 days) followed by oral anticoagulant therapy for at least 3-6 months, or longer in the case of a persistent underlying risk factor (e.g., life-long in the setting of congenital anticoagulant deficiency).

It would be further of use for treating physicians to be able to quickly and accurately monitor a patient's total clot formation and lytic activity, both lysis resulting from natural fibrinolytic activity and from physiological responses to the therapeutic administration of thrombolytic agents. It would also be of use to distinguish changes to properties of clotted blood caused by lytic activity from those caused by therapeutically administered agents or by pathological conditions, including disseminated intravascular coagulation. In order to monitor blood condition changes caused by lytic activity, a test which evaluates changes to a sample of clotted blood in which lysis is allowed to proceed would prove useful. However, the present standard for fibrinolytic assessment, the euglobulin clot lysis assay (ECLA), also referred to as euglobulin lysis time (ELT), only permits the evaluation of those changes after key inhibitors of fibrinolysis have been removed from the plasma.

Physicians have been hindered by an inability to prescribe individualized doses of thrombolytic or anti-fibrinolytic agents tailored to the unique physiological responses of a particular subject. Currently, no known tests are commercially available to determine the dose response to thrombolytic and anti-fibrinolytic agents. In the absence of such dose response data, a standardized dose is usually prescribed. A standardized dose may be either inadequate or excessive for a particular patient because of variations in body size, blood volume, blood chemistry, physiologic response and pathological or surgical conditions. Thus, a rapid test to assess the formation of a clot and the lysis of a clot over a given time would be very useful for diagnosis and therapeutic monitoring.

### CloFAL Assay

A non-limiting example of a Clot Formation and Lysis (CloFAL) assay may utilize a buffered reactant solution containing trace amounts of one or more activators of coagulation, such as calcium, tissue factor (TF) and/or thrombin, and one or more activators of clot lysis, such as tissue-type plasminogen activator (tPA) (preferably, two-chain recombinant human tPA). TF (preferably recombinant human TF) may be used in lipidated form for platelet-poor plasma assay or in non-lipidated form for platelet-rich plasma assay. An exemplary buffer solution may comprise Tris-buffered saline solution with calcium chloride.

The buffered reactant solution may be added to a sample, such as fresh or freeze-thawed, platelet-poor or platelet-rich plasma in triplicate or quadruplet wells of a 96-well assay plate. Samples may also include a blank well containing only reagent for comparison with the test samples. Samples may further comprise one or more cellular entities, such as white blood cells and/or endothelial cells, in suspension or in a monolayer. The plate may be analyzed in an automated, thermoregulated (37° C.) spectrophotometer and the course of clot formation and subsequent lysis may be monitored as continuous changes in the absorbance of the specimen over a course of time, for example, over three hours. In a preferred embodiment, optical density at 405 nm or dual wavelength OD (405 and 630 nm) may be monitored continuously or at selected frequent time intervals. The spectrophotometer preferably is interfaced with a computer to permit analysis of kinetic OD measurements using (a) data analysis program(s). A curve may be generated over the course of the assay reactions that include an initial baseline OD, followed by a progressive rise in optical density to a point of maximum OD, then completed by a progressive decline in optical density to baseline. A plasma standard (preferably pooled plasma from healthy individuals) and controls (preferably one normal and one to two abnormal controls) may be run simultaneously with the clinical/laboratory sample(s) using the same protocol.

A clotting curve may be generated whereby coagulation and fibrinolytic parameters of the plasma sample are obtained, relative to a simultaneously run pooled normal subject plasma standard. Specific measurements may include the lag time (the time from assay initiation to time to clot initiation, as measured by rise in OD above baseline or a specified threshold), the maximum amplitude (MA) (maximum OD minus baseline OD), the time to maximum turbidity (T₁), the time to completion of the first phase of decline in turbidity (T₂), and the area under the curve (AUC) over the course of the measured time intervals. A coagulation index (CI) may be calculated, in one example, as the AUC over the course of the first 30 minutes of an assay, referenced to a plasma standard. A fibrinolytic index (FI) may be calculated, for example by relating the ratio of T2 to T1 for a sample as compared to a standard, with a correction factor for differences in maximum OD, as discussed below. Alternatively, an FI may be calculated by the area above the curve, or a reciprocal AUC, from T1 to T1+30 minutes for a sample compared to a standard, with a correction factor as above. Specimens may be compared between normal controls and patients suspected of having, or known to have, one or more pathologic conditions, such as hemophilia or other diseases relating to clotting and or clot lysis.

Particular details of exemplary embodiments of CloFAL assays are provided in the Examples below. However, the skilled artisan will realize that the concentrations of various reagents and times and temperatures of reactions may be varied from those specified below without undue experimentation by the person of ordinary skill in the art. Further, where various factors, such as calcium, TF and tPA are disclosed, such factors may be substituted or supplemented with alternative factors known in the art to exhibit similar activities, within the scope of the claimed methods and compositions. The CloFAL global assay is reproducible and analytically sensitive to deficiencies and excesses of key components in the coagulation and fibrinolytic systems, as well as to physiologic alterations in hemostasis. The measurement of these parameters may be applied to assess subjects with known and/or as yet undefined hemorrhagic and prothrombotic conditions.

In one embodiment, any of the combination clot formation and fibrinolysis assay results may be analyzed in an individual suffering from a heart condition. Non-limiting examples of heart conditions include but are not limited to myocardial ischemia, myocardial infarction, acute coronary syndromes, atherosclerotic coronary artery disease, valvular disease, and congestive heart failure.

In another embodiment, any of the combination clot formation and fibrinolysis assay results may be analyzed in an individual suffering from a prothrombotic condition. Examples of prothrombotic conditions include but are not limited to venous or arterial thromboembolism, including stroke, as well as hypercoagulable states (in particular, factor V Leiden and prothrombin 20210 mutations, antiphospholipid antibodies, anticoagulant deficiency, and elevated levels of procoagulant factors, homocysteine, or lipoproteins).

In certain embodiments, any of the combination clot formation and fibrinolysis assay results may be analyzed in an individual suffering from a bleeding condition. Non-limiting examples of bleeding conditions include the hemophilias and other coagulation factor deficiencies or dysfunctions (including a/hypo/dysfibrinogenemia), von Willebrand disease, platelet function abnormalities and fibrinolytic abnormalities (e.g., PAI-1 deficiency).

In yet another embodiment, any of the combination clot formation and fibrinolysis assay results may be analyzed in healthy children and adults to assess bleeding and/or prothrombotic risk in the steady state and in times of altered (pathologic or physiologic) hemostasis, including the special physiologic states of pregnancy and the neonatal period. Any combination of clot formation and fibrinolysis assay may be used as a pre-operative or pre-treatment screening test on a sample from a test subject. In addition, any combination of clot formation and fibrinolysis assay may be used as a post-operative or post-treatment test on a sample from a test subject.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Subject Groups

All healthy individuals recruited for the establishment of physiologic CloFAL assay values (i.e., children, pregnant women at term, and term neonates) were without prior bleeding or thrombotic histories and were not receiving anticoagulant, anti-platelet, or estrogen-containing medications. These criteria were also applied for a group of healthy adults from whom plasma was obtained commercially (Core Set Adult Normals, George King Bio-Medical, Inc., Overland Park, Kans.). The median age of healthy children (n=22) was 11 years (range: 5-18 years), of adults (n=22) was 39 years (range: 21-52 years), and of pregnant women (n=24) was 24 years (range: 19-39 years).

### Blood Collection and Sample Processing Procedures

Blood was collected with the child or adult participant at rest in the seated position by atraumatic peripheral venipuncture technique with minimal applied stasis. Samples were collected into BD Vacutainer, 3.2% buffered sodium citrate, siliconized blood collection tubes (Becton-Dickinson, Franklin Lakes, N.J.), with collection of the initial 1 mL of blood into a discard tube. In the case of neonates, cord blood was collected via the dual-clamp two-syringe technique, as previously described (Goodnight & Hathaway, 2001). All specimens were centrifuged for 15 minutes at 4° C. and 2500×g, and the plasma supernatant was then centrifuged for an additional 15 minutes to remove any residual platelets. All samples were aliquoted into 1.5 mL copolymer polypropylene long-term freezer storage tubes with O-ring screw caps (USA Scientific, Ocala, Fla.) and stored at −70° C. until time of assay. Storage time was studied up to six months in five healthy individuals, with no change in the CloFAL curve observed over this time period. Commercially-obtained individual and pooled platelet-poor plasma specimens (George King Bio-Medical, Inc., Overland Park, Kans.) were collected and processed by a similar protocol.

### CloFAL Assay Procedure

The assay described here was modified from those of He et al. (1999) and Smith et al. (2003). As compared to that by Smith et al., which evaluates only fibrinolysis, the CloFAL assay permits assessment of coagulability as well. Furthermore, when compared to the global assay of He et al., the CloFAL assay permits testing with a single reagent to evaluate both coagulation and fibrinolysis, rather than requiring (as does that of He et al.) the preparation of two distinct reagents for separate evaluation of the plasma sample. In addition, unlike the assay of He et al., the CloFAL assay does not require the use of thrombin (a key end-product of the coagulation reactions) among the assay reagents. Frozen plasma aliquots were thawed in a 37° C. water bath for three minutes. Comparison of freeze-thawed versus fresh platelet-poor plasma specimens from the same individual have revealed no differences in the CloFAL curve. Plasma samples (fresh or freeze-thawed) were maintained for up to 30 minutes in an ice-water bath until time of assay. For preparation of reactant solution, recombinant lipidated human TF (American Diagnostica, Stamford, Conn.; 0.5 μg/mL stock solution prepared according to manufacturer instruction) and two-chain recombinant tPA (American Diagnostica, Stamford, Conn.; 0.5 mg/mL) were added to a stock solution of Tris-buffered saline (TBS; 66 mM Tris, 130 mM NaCl, pH=7.0) containing 34 mM CaCl₂, to a concentration of 10 pM and 900 ng/mL, respectively (final concentrations of 5 pM TF and 450 ng/mL tPA after addition of reactant solution to plasma sample, as described below). TBS stock solutions were stored for up to one month at 4° C., and reconstituted stock solutions of tPA and TF were stored for up to one month (and at least 24 hours) at −70° C., for use in preparation of fresh reactant solution. The reactant solution was maintained at room temperature until time of assay, not to exceed 30 minutes.

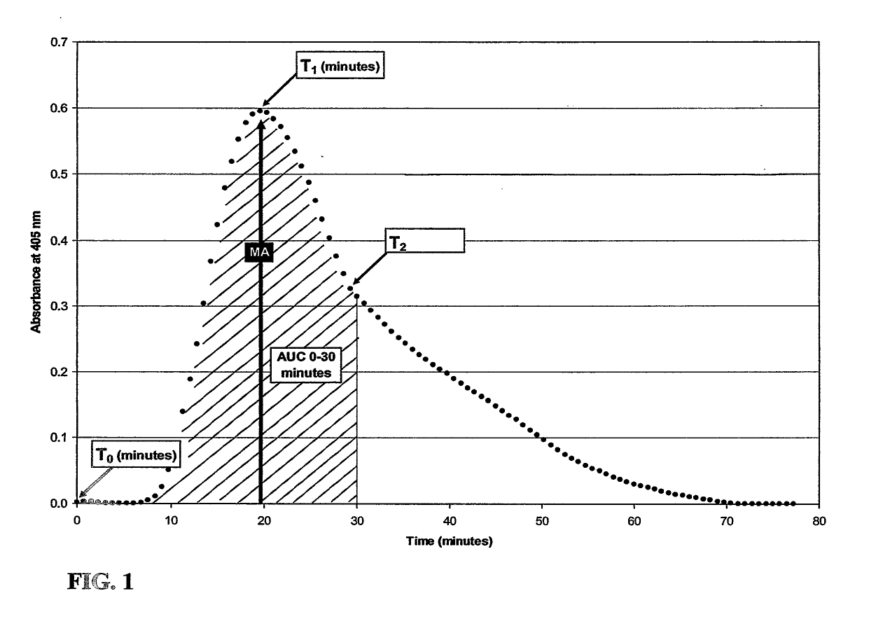

For each patient sample to be analyzed, 75 μL of freeze-thawed or fresh plasma was dispensed into each of three wells in a round-bottom, 96-well, Nunc assay plate (Fisher Scientific, Santa Clara, Calif.), and then pre-warmed at 37° C. for three minutes. Using a multi-tip automated pipette, 75 μL of reactant solution was added simultaneously to each well. The plate was then immediately placed in an eight-channel microplate spectrophotometer (PowerWave HT, Bio-Tek Instruments, Winooski, Vt.) for dual kinetic absorbance measurements at 405 nm and 630 nm at 45-second intervals for 3 hours, following an initial five-second mixing step prior to the first reading. The spectrophotometer interfaced with a computer such that all its operations, including continuous analysis of delta-absorbance (405 nm minus 630 nm) data using KC4™ PC software, may be automated. As shown in FIG. 1, beginning at time zero (T₀), a curve was generated over the course of the assay reactions that had an initial baseline absorbance, followed by a progressive rise in absorbance to a point of maximum absorbance (achieved at T₁), then a first phase of decline in absorbance (ending at T₂, the time point at which the slope of decline in absorbance changes by +0.10 mOD/min), and completed by a further decline in absorbance to baseline.

The kinetic absorbance data was exported to Microsoft Excel and absorbance measurements at each time point were averaged for the triplicate runs of each specimen. Using the averaged absorbance for the specimen, the maximum amplitude of rise in absorbance was determined (MA=maximum absorbance minus baseline absorbance, where baseline absorbance was obtained by averaging the third through eighth kinetic readings). T₁ and T₂ were also obtained. In one example using the area under the curve (AUC) over the course of the initial 30 minutes of the assay, a coagulation index (CI) was calculated that relates this value for the sample to that of the standard run with each assay (FACT, George King Biomedical, Overland Park, Kans.), as follows:

$$CI = \frac{{( {AUC}_{0 \text{-} 30 min} )}_{sample}}{{( {AUC}_{0 \text{-} 30 min} )}_{standard}} \times 100$$

A fibrinolytic index (FI) was calculated by relating the ratio of the time to completion of the first phase of decline in absorbance (T₂) to the time to maximum absorbance (T₁) for the sample as compared to the standard, with a correction factor for differences in maximum absorbance (MA_{standard}/MAₛₐₘₚₗₑ), as follows:

$$FI = \frac{T 2 / {( T 1 )}_{sample}}{T 2 / {( T 1 )}_{standard}} \times \frac{{MA}_{standard}}{{MA}_{sample}} \times 100$$

This formula can be simplified to:

$$FI = \frac{T 2 / {( T 1 * MA )}_{sample}}{T 2 / {( T 1 * MA )}_{standard}} \times 100$$

In summary, the CloFAL curve of each plasma specimen was analyzed for MA, T₁, T₂, CI, and FI.

### Abnormal/Altered Plasma Experiments

Factor VIII deficient plasma was obtained from a patient with severe congenital deficiency, with a measured factor VIII activity of <1 U/dL. All other specific factor-deficient human plasmas were obtained commercially as snap-frozen specimens from patients with congenital factor deficiencies (Factor II, V, VII, IX, X, XI, XII, XIII, Prekallikrein, High-Molecular-Weight Kininogen [HMWK], and Fibrinogen Deficient Plasmas, George King Bio-Medical, Inc., Overland Park, Kans.). The activity level of the deficient factor was assayed at <1 U/dL in all cases, with the exception of factor II activity and fibrinogen concentration, which were 3 U/dL and 8 mg/dL, respectively. To test the analytic sensitivity of the assay to fibrinogen and factor VIII, fibrinogen-deficient plasma was mixed with standard normal pooled plasma to achieve final concentrations of 8, 81, 125, 164, and 212 mg/dL, and factor VIII-deficient plasma was serially diluted with standard normal pooled plasma to achieve final concentrations of <1, 6, 13, 50, and 100 U/dL.

In the altered fibrinolysis studies, TAFI activation was blocked in order to enhance fibrinolysis by adding potato tuber carboxypeptidase inhibitor (PTCI; Sigma-Aldrich, Inc., Saint Louis, Mo.) to standard normal pooled plasma to achieve a final plasma PTCI concentration of 50 μg/mL. To inhibit fibrinolysis, standard normal pooled plasma was treated with aminocaproic acid to achieve a final plasma concentration of 2.5 mg/mL. The effect of PAI-1 deficiency was examined using a plasma sample obtained 24 hours following a therapeutic dose of aminocaproic acid from a patient with congenital PAI-1 deficiency (PAI-1 antigen level, 0 ng/mL).

In the heparin studies, porcine unfractionated heparin sodium (Hep-Lock, Elkins-Sinn, Inc., Cherry Hill, N.J.) was added to standard normal pooled plasma to achieve final plasma heparin concentrations of 2 U/mL, 1 U/mL, 0.5 U/mL, 0.1 U/mL, and 0.05 U/mL, respectively. For heparin reversal and the heparinase control, 6 mg of heparinase (Dade® Hepzyme® Reagent, Dade Behring Inc., Newark, Del.) was dissolved in 0.25 mL of plasma sample, as previously described (Manco-Johnson et al 2000).

### Correlative Laboratory Assay Procedures

Prothrombin times (PT) were measured using Simplasting Excel, and activated partial thromboplastin times (aPTT) using 0.025 molar calcium chloride and Automated APTT® reagent (bioMerieux, Inc., Durham, N.C.). Plasma fibrinogen concentration was determined by the clotting method of Clauss using Dade Behring thrombin and calibration reagents (Dade Behring, Marburg, Germany). Plasma factor VIII activity levels were ascertained with standard one-stage clotting assay with the same reagents as above for aPTT. All of these clotting assays were performed on an ST4 coagulometer (Diagnostica Stago, Asnieres-sur-Seine, France). ELT was performed using the automated euglobulin clot lysis assay developed in our laboratory, as described previously (Smith et al., 2003).

### Statistical Analysis

Median values of laboratory test results were compared by Wilcoxon rank sum test. Spearman correlation was used to test for associations among laboratory test results. For all analyses, SAS statistical software was used (SAS Institute, Cary, N.C.), with a P-value of <0.05 considered as statistically significant.

### Example 1

### CloFAL Clot Formation and Lysis

FIG. 1 illustrates a non-limiting example of a typical CloFAL clot formation and lysis curve for a healthy adult. The exemplary analytical technique involves a standard normal pooled adult platelet-poor plasma specimen. The intra-assay coefficients of variation (CV) for the CloFAL assay were established for a normal control by using this standard along with 25 repeated samples of normal pooled plasma from a different pool of healthy individuals (Pooled Normal, George King Biomedical, Overland Park, Kans.), and for an abnormal control using 30 repeated samples of multi-factor reduced plasma standard (B-FACT, George King Biomedical, Overland Park, Kans.). In each case, plasma samples were analyzed in triplicate on the same assay plate in a single run. Intra-assay CVs for normal controls were MA 2.5%, T₁ 8.7%, T₂ 8.70%, CI 5.0%, and FI 12.8%, and for abnormal controls were MA 6.9%, T₁ 5.5%, T₂ 4.2%, CI 18.6%, and FI 7.8%. Inter-assay CVs, determined via serial testing of the normal and abnormal standards on 20 separate runs, were MA 5.3%, T₁ 14.8%, T₂ 15.5%, CI 14.20%, and FI 8.3% for normal controls, and 8.8%, T₁ 5.5%, T₂ 4.1%, CI 18.1%, and FI 20.1% for abnormal controls.

### Example 2

### Comparative CloFAL Curves

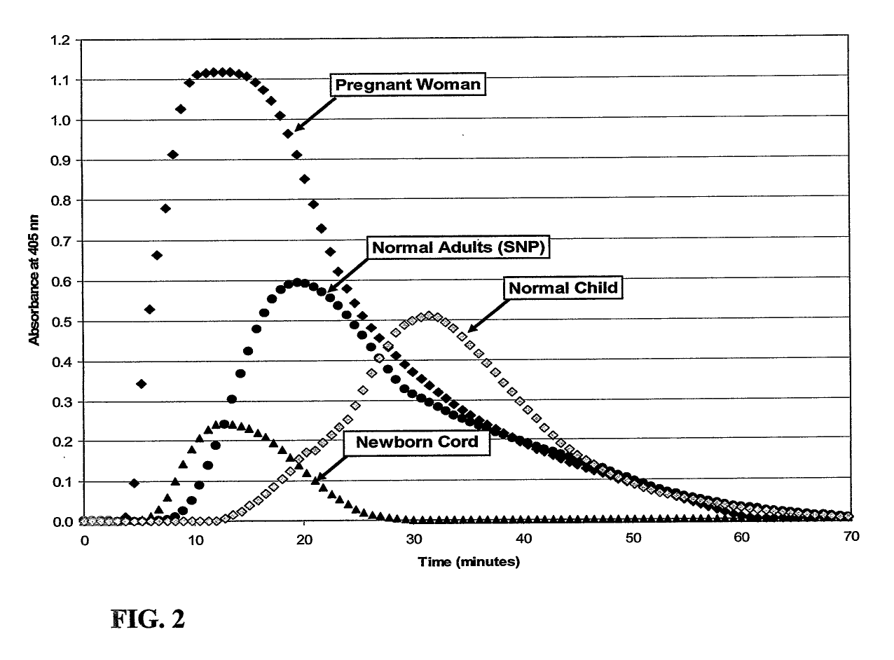

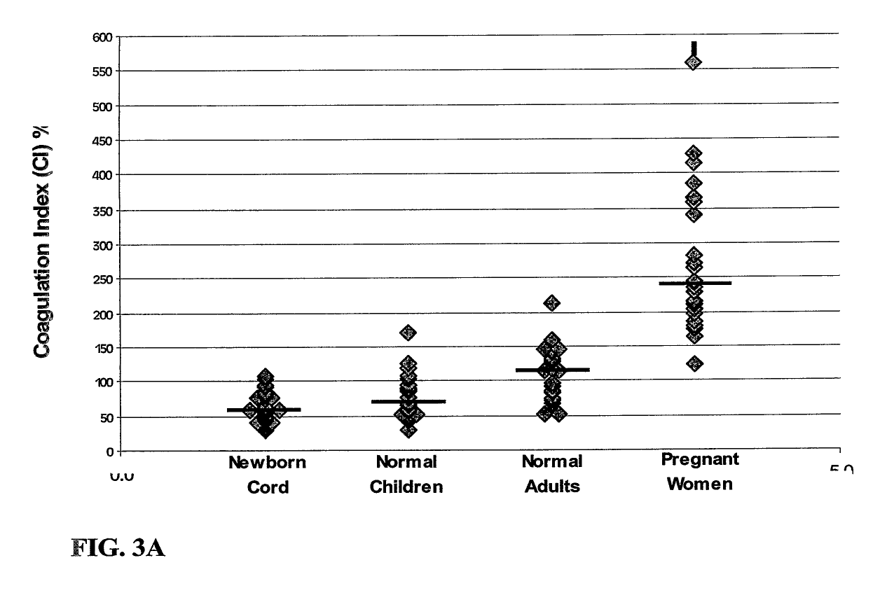

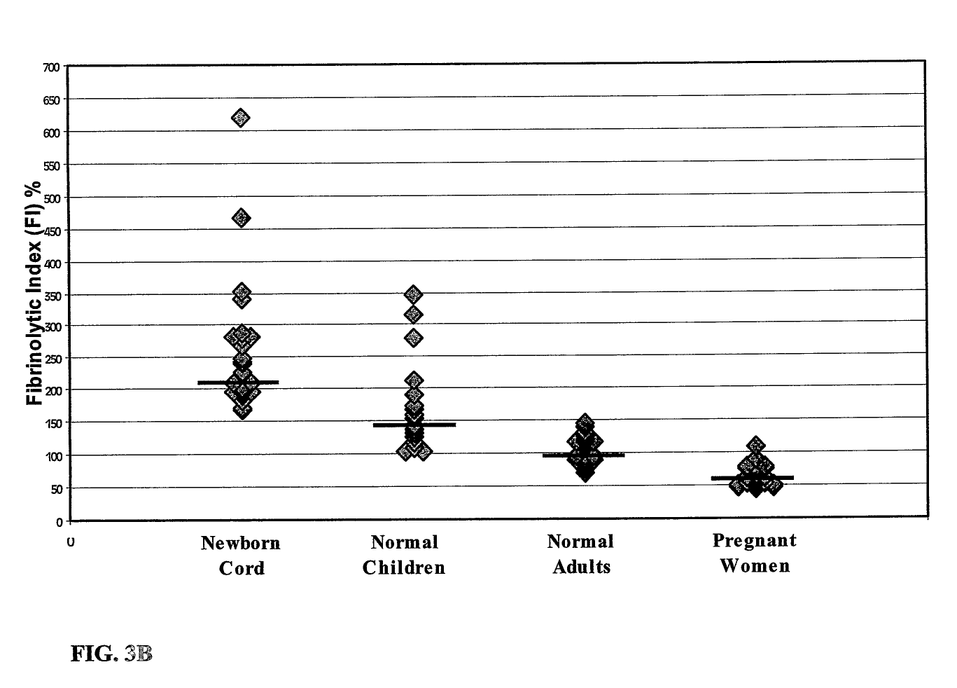

Physiologic ranges for CloFAL parameters were determined in healthy adults (n=22) and children (n=22), as well as healthy pregnant women (n=24) and neonates (n=27). FIG. 2 illustrates a non-limiting example of CloFAL curves from healthy adults, a newborn cord, and a pregnant woman. Tables 1a and 1b provide median CloFAL CI and FI values, PT, aPTT, factor VIII activity, fibrinogen concentration, PAI-1 antigen and activity, and automated ELT for each of the four subject groups. The scatter-plots of FIGS. 3A and 3B comparatively display the distribution of CI and FI values by group. Median CI was significantly decreased, while FI was markedly increased, in neonates as compared to healthy adults (CI: 58% vs. 115%, FI: 210% vs. 95%; P<0.001 for each). These findings were in contrast with those of healthy pregnant women, in whom median CI was notably increased, and FI decreased, when compared with adults (CI: 239% vs. 115%, FI: 59% vs. 95%, P<0.001 for each). When comparing healthy adults and children, CI was significantly higher among adults, while FI was greater among children (CI: 115% vs. 73%, P=0.01; FI: 95% vs. 140%; P<0.001).

### Example 3

### Effect of Deficiencies of Coagulation Factors and Fibrinolytic Regulators on CloFAL Components

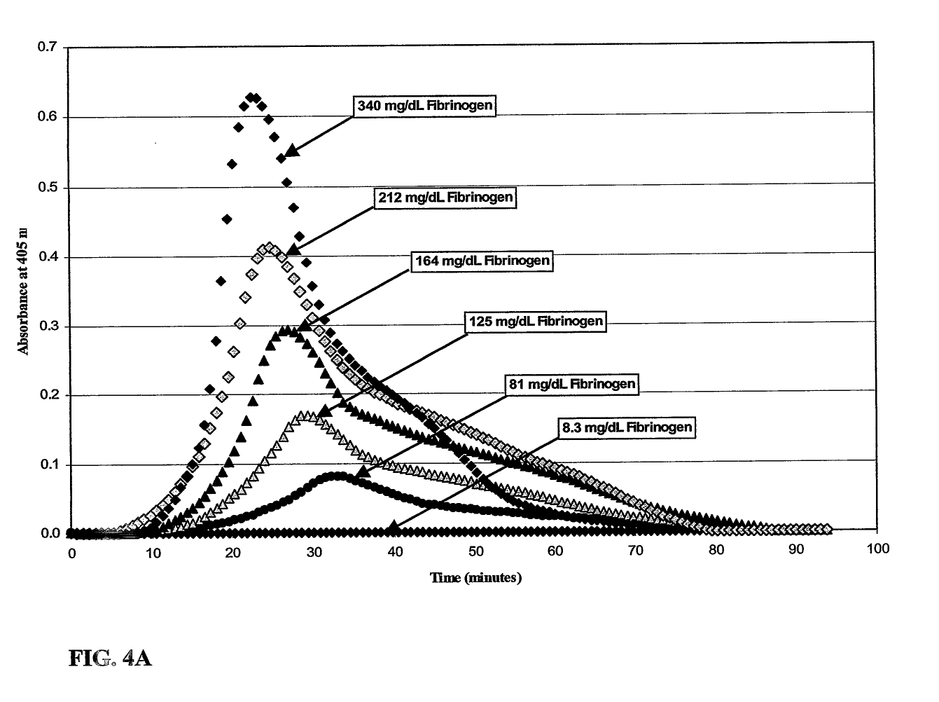

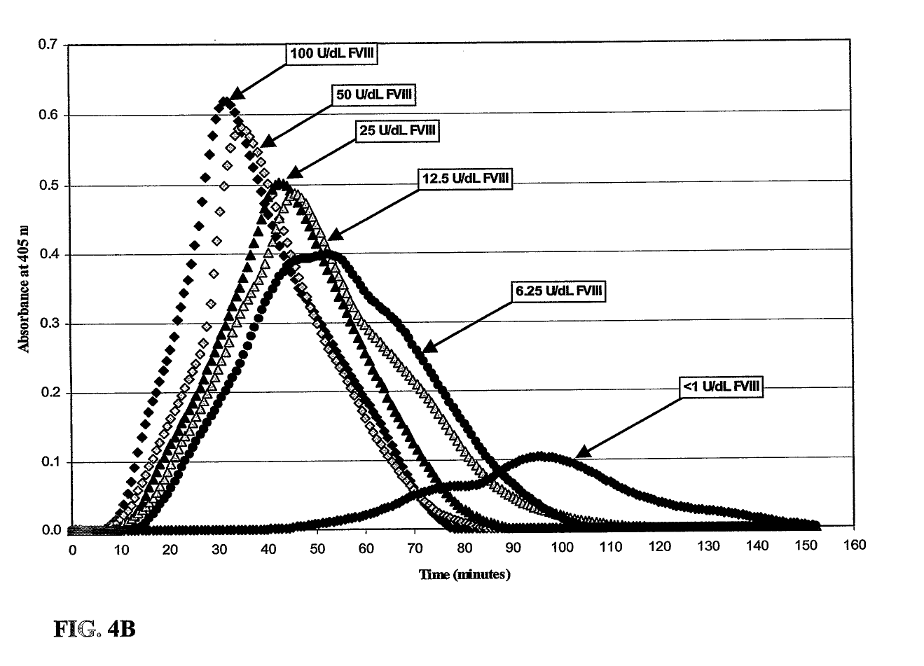

FIGS. 4A and 4B illustrate a non-limiting example of the concentration effects of fibrinogen and factor VIII. Fibrinogen and factor VIII influence MA and T₁ (and hence CI) in a concentration-dependent manner. The exemplary analytical technique analyses the influence of deficiencies of coagulation factors and fibrinolytic regulators upon CloFAL components. Patient plasmas deficient in fibrinogen, factors II, V, VII, VIII, IX, X, XI, XII, or XIII, prekallikrein or HMWK were also investigated. In addition, standard normal pooled plasma was treated with PTCI or aminocaproic acid in order to examine the effects of enhancement or inhibition of fibrinolysis, respectively.

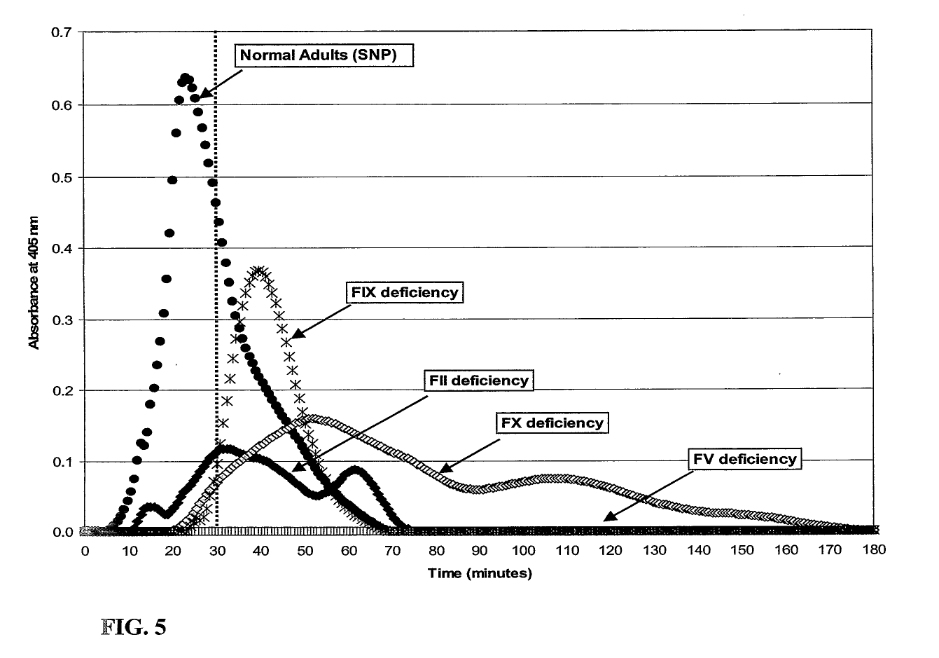

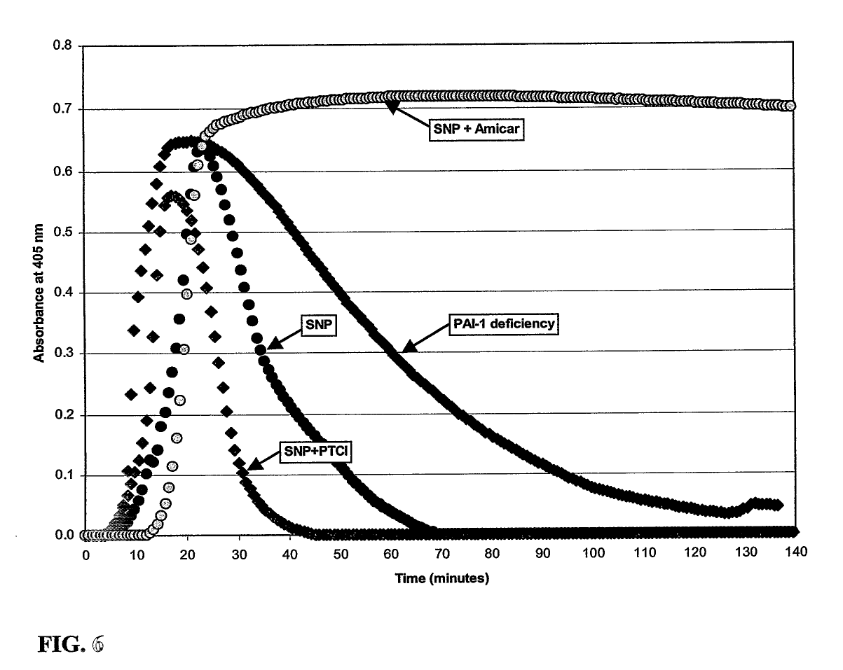

Table 2 and FIG. 5 illustrate a non-limiting example of CloFAL values and curves, respectively, for numerous altered coagulation conditions, and demonstrate that the greatest impact upon the absorbance, and the resultant CI, occurs with severe deficiency of fibrinogen or factors II, V, VII, VIII, IX, or X. The exemplary analytical technique illustrates the results of the altered fibrinolysis studies in FIG. 6. In these experiments, the duration of the first phase of decline in turbidity in the CloFAL curve is prolonged by TAFIa inhibition and PAI-1 deficiency, resulting in an increased FI. By contrast, there is no decline in absorbance, and hence FI is zero, in the setting of aminocaproic acid treatment.

### Example 4

### Heparin Effects

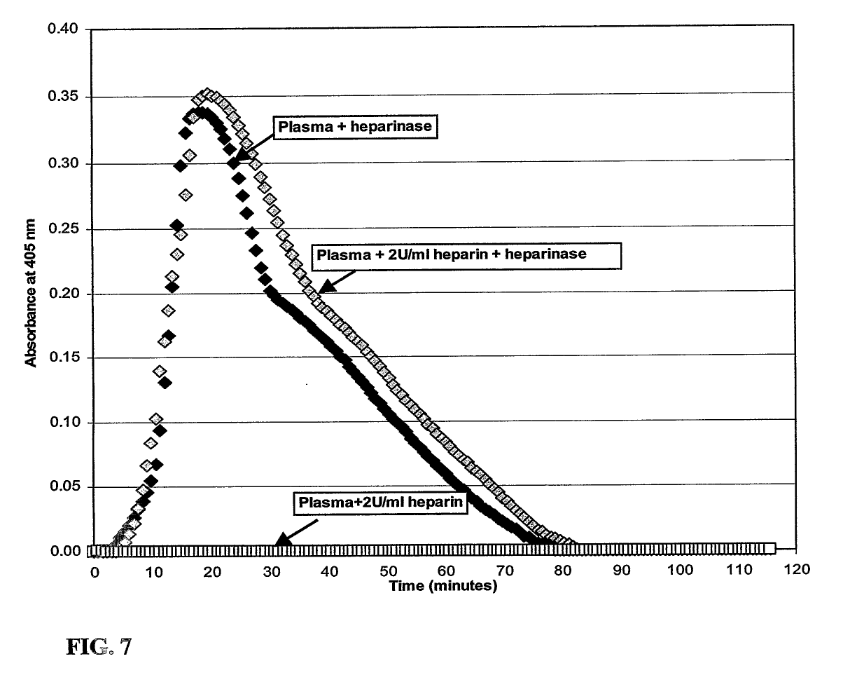

FIG. 7 illustrates a non-limiting example of the sensitivity of the CloFAL assay to various concentrations of heparin. The exemplary analytical technique illustrates the degree to which any influence of heparin could be ablated by heparinase treatment of specimens prior to assay. As shown in FIG. 7, the presence of heparin at 2 U/mL greatly prevented the rise in absorbance of the CloFAL curve (indeed, prolongation and attenuation of the rise in absorbance occurred with heparin concentrations of as little as 0.1 U/mL), and this effect was reversible by heparinase treatment of samples prior to assay.

### Example 5

### Statistical Analyses

The statistical relationship was explored between CloFAL values and various markers and components of coagulation and fibrinolysis across individuals in all four subject groups. There was a positive correlation of CI with factor VIII activity (i.e., as factor VIII increased, so did CI; r=0.62, P<0.001) and even more so with fibrinogen concentration (r=0.79, P<0.001). Conversely, CI correlated negatively with PT and aPTT (i.e., as PT and aPTT increased, CI decreased; r=−0.52, P<0.001 and r=−0.44, P<0.001, respectively). In addition, FI correlated negatively with both PAI-1 antigen and activity (i.e., as PAI-1 antigen and activity increased, FI decreased; r=−0.61, P<0.001 and r=−0.67, P<0.001) and, to a slightly greater extent, with automated ELT (r=−0.69, P<0.001). FI also correlated negatively with fibrinogen concentration, but this association was not strong (r=−0.33, P=0.001).

Using MA, T₁, and CI measurements it was discovered that these parameters were dependant upon fibrinogen and plasma levels of procoagulant factors. On the other hand, FI is affected by TAFIa. Median CI was significantly decreased, while FI was markedly increased, in term neonates as compared to healthy adults (CI: 58% vs. 115%, FI: 210% vs. 90%; P<0.001 for each). These findings were in contrast with those of healthy pregnant women, in whom median CI was notably increased, and FI decreased, when compared with adults (CI: 239% vs. 115%, FI: 59% vs. 90%; P<0.001 for each).

### Example 6

### Additional Protocols

In the following Examples, plasma from healthy children and adults versus children and adults with factor VIII deficiency was examined, as well as the plasma coagulative response to administration of factor VIII replacement therapy in patients with severe hemophilia A. Modifications to protocols were as indicated below.

### Subject Groups

Healthy subjects included those without personal or first-degree family history of bleeding or thrombosis, were not taking any medications, and had no acute infection or chronic illness. Apparently-healthy individuals with abnormal prothrombin times or activated partial thromboplastin times were excluded from the analysis. Plasma from healthy adults was obtained commercially (Core Set Adult Normals, George King Bio-Medical, Inc., Overland Park, Kans., USA). The median age of healthy adults (n=25) was 35 years (range: 21-53 years) and of healthy children (n=47) was 5 years (range: 13 months-17 years). In both the healthy and FVIII-deficient groups, children were defined as individuals less than or equal to 18 years of age.

Children and adults with FVIII deficiency were without exogenous FVIII treatment within the prior 96 hours. Other excluded factors included use of other medications that affect hemostasis (e.g., estrogens, non-steroidal anti-inflammatory drugs, anti-fibrinolytic agents), evidence of active hepatitis, or signs and symptoms of acute infection. Severe, moderate, and mild deficiencies of FVIII were defined as baseline values of FVIII activity less than 1 U/dL, between 1 and 5 U/dL, and greater than 5 U/dL, respectively, according to classical laboratory criteria (DiMichele, 2001). Patients with recent or current evidence of inhibitory antibodies to FVIII were excluded from the analysis.

### Blood Collection and Sample Processing Procedures

Blood was collected by atraumatic peripheral venipuncture technique with minimal applied stasis into BD Vacutainer 3.2% buffered sodium citrate siliconized blood collection tubes (Becton-Dickinson, Franklin Lakes, N.J., USA), with the participant at rest and alert in a seated position, or in the recumbent position following inhaled anesthesia for elective surgery. The initial 1 mL of blood was collected into a discard tube. Platelet-poor plasma was obtained within 45 minutes of collection via initial centrifugation of the whole blood specimens at 4° C. and 2500×g for 15 minutes, followed by re-centrifugation of the plasma supernatant for 15 minutes at the same settings. Platelet-poor plasma aliquots were frozen and stored at −70° C. in polypropylene long-term freezer storage tubes. Commercially-obtained platelet-poor plasma specimens had been collected, processed, and stored using the same protocol.

### CloFAL Assay Technical Procedure

For each patient specimen, 75 μL of platelet-poor plasma was loaded in quadruplicate wells of a 96-well Nunc microassay plate (Fisher Scientific, Santa Clara, Calif.). Next, 75 μL of Tris-buffered saline (TBS; 66 mM Tris, 130 mM NaCl, pH=7.0; first well) or reagent (TBS with 34 mM CaCl₂, 10 pM recombinant lipidated human tissue factor (American Diagnostics, Stamford, Conn., USA) and 900 ng/mL recombinant two-chain human tissue-type plasminogen activator; remaining wells) was added. Kinetic absorbance measurements were obtained at 405 nm and 630 nm at 45-second intervals in a PowerWave HT™ microplate scanning spectrophotometer (BIO-TEK Winooski, Vt., USA) for 3 hours. A turbidimetric fibrin clot formation and lysis curve was generated, from which a coagulation index was calculated with respect to the plasma standard (FACT, George King Biomedical, Inc., Overland Park, Kans.), based upon the area under the curve at 30 minutes. Various fibrinolytic indices were also determined in reference to the plasma standard.

### Correlative Laboratory Assay Procedures

Levels of aPTT were determined on an ST4 coagulometer (Diagnostica Stago, Asnieres-sur-Seine, France) using 0.025 M CaCl₂ and Automated APTT® reagent (BioMerieux, Inc., Durham, N.C., USA). Factor VIII activity was measured by one-stage clotting assay with the same reagents as for aPTT. Von Willebrand factor antigen (vWF Ag) was determined by ELISA using the REAADS® kit (Corgenix, Westminster, Colo., USA), using spectrophotometric detection of vWF-bound anti-vWF primary antibody at 450 nm with a horseradish peroxidase/anti-human vWF secondary antibody conjugate and teramethylbenzidine/H₂O₂ substrate.

### Clinical Bleeding Severity Assessment

Bleeding severity was assessed by clinical history as modified from previously-published standardized criteria (DiMichele, 2001). This assessment was performed by a single clinician who was blinded to the results of the CloFAL assay. Rating of severe versus non-severe hemophilia A utilized the data from this assessment, and was performed by a different single clinician who was blinded to patient identities and laboratory values.

### Statistical Methods

Statistical analyses were performed using SAS software (SAS Institute, Cary, N.C., USA). For all hypothesis testing, a P-value of <0.05 was considered statistically significant. Median values were compared between groups by Wilcoxon rank sum test and correlations between laboratory assay results were evaluated using the Spearman rank correlation test. Clinical sensitivity was calculated as the number of true-positive test results divided by the sum of true-positives and false-negatives. Normal values for aPTT and factor VIII activity in adults and children were based upon reference ranges established the same laboratory in healthy individuals (adults, n=65 and n=62, respectively; children, n=56 for each) using the aforementioned methodologies. Reference ranges for CloFAL assay values were calculated separately for adults and children of the healthy subjects groups, as the median +/−(1.25*interquartile range).

### Example 7

### Effect of Factor VIII Levels on CloFAL Measurements and Correlative Coagulation Laboratory Results, and Correlation with Clinical Bleeding Severity

Table 3 shows exemplary distributions of age and laboratory and clinical disease severities for individuals with factor VIII deficiency, as well as distributions of age for healthy subjects.

Table 4 shows exemplary median values for CI, T₁, MA, aPTT, one-stage FVIII assay, and vWF Ag ELISA for pediatric and adult groups with and without factor VIII deficiency.

Table 5 shows a non-limiting example of the sensitivities of the CloFAL global assay and aPTT for different levels of severity of factor VIII deficiency.

### Statistical Analyses

Among adults and children, the median age of subjects did not differ significantly between healthy and FVIII-deficient groups. The CloFAL assay coagulation index (CI), a measure of the area under the clotting curve, was significantly reduced in the FVIII-deficient groups when compared to the healthy groups (adults: 1% vs. 94%, respectively, P<0.001; children: 5% vs. 71%, P<0.001). In addition, the time to maximal amplitude (T₁) of the clotting curve in the CloFAL assay was significantly prolonged in FVIII-deficient subjects when compared to healthy controls (adults: 48.8 vs. 25.5 minutes, P<0.001; children: 67.5 vs. 33.4 minutes, P<0.001). Similarly, the aPTT was significantly prolonged in the FVIII-deficient groups when compared to the healthy subjects (adults: 53.2 vs 37.1 seconds, P<0.001; children: 54.7 vs. 39.1 seconds, P<0.001). Interestingly, the CloFAL CI correlated at least as strongly with factor VIII activity by one-stage clotting assay (r=0.75, P<0.001) as did the aPTT (r=−0.69, P<0.001).

Using the coagulation parameters of CI and T₁, the sensitivity of the CloFAL assay for mild FVIII deficiency (i.e., classical laboratory designation) was 94%, while that of the aPTT was 88%. Similarly, the CloFAL assay was found to be superior to the aPTT in its sensitivity (96%) for clinically-defined mild hemophilia A, using standardized bleeding criteria.

### Example 8

### Comparative CloFAL Curves for Monitoring Plasma Coagulative Response Following Factor VIII Infusion in Hemophilia A

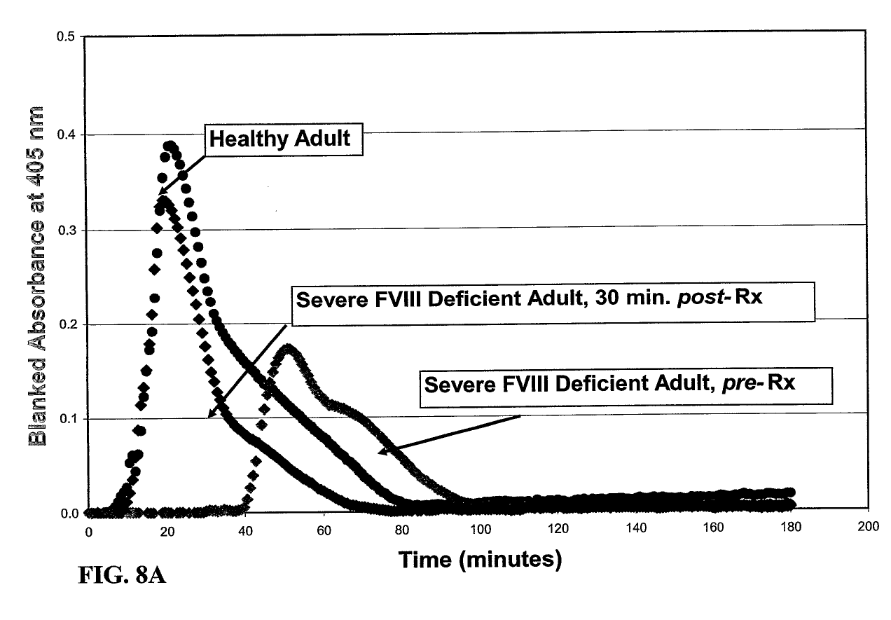

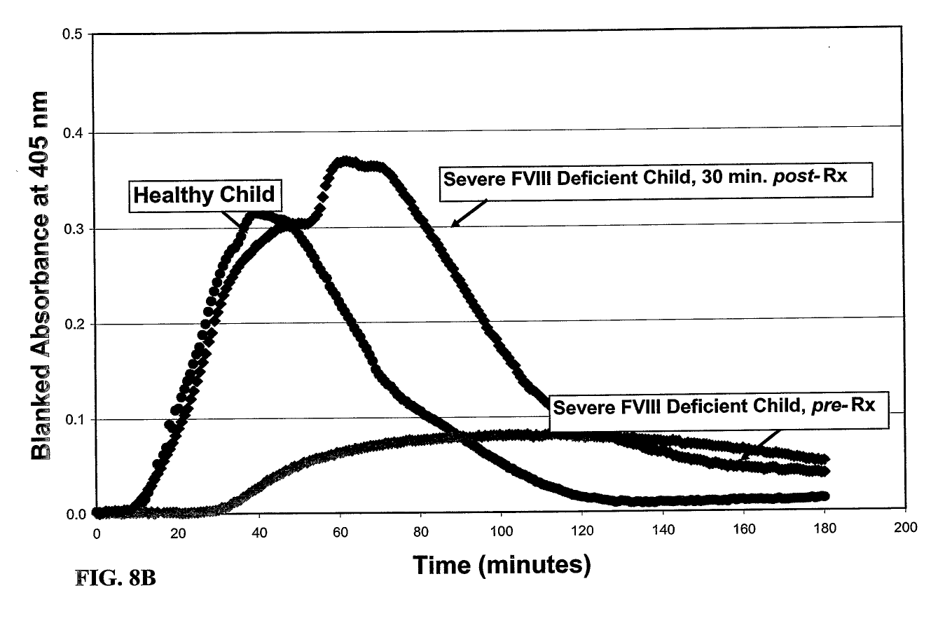

FIGS. 8A and 8B show representative examples of the hemostatic response to therapeutic or prophylactic recombinant human FVIII administration in severe hemophilia A, as measured by the CloFAL global assay. Following FVIII infusion, the CloFAL waveforms became substantially normalized, with considerable increase in maximum amplitude and decrease in T₁. Accordingly, in the adult patient (FIG. 8A), 30 minutes following a 55 U/kg dose of FVIII, the CloFAL CI had increased from 0% pre-infusion (48 hours following the last FVIII dose) to 85% post-infusion. In the pediatric patient (FIG. 8B), the CloFAL CI increased from a pre-infusion value of 0% (48 hours following the last FVIII administration) to a post-infusion value of 63%, 30 minutes following a 26 U/kg dose of FVIII. In both cases, post-infusion CI rose to within normal limits, as established in the corresponding adult or pediatric healthy subject group.

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES CITED

Andrew M, Vegh P, Johnston M, et al. Maturation of the hemostatic system during childhood. Blood 1992; 80:1998-2005.
Antovic A, Blombäck M, Bremme K, et al. Increased hemostasis potential persists in women with previous thromboembolism with or without APC resistance. J Thromb Haemost 2003b; 1:2531-2535.
Antovic J P, Yngen M, Östenson C-G, et al. Thrombin activatable fibrinolysis inhibitor and hemostatic changes with type I diabetes mellitus with and without microvascular complications. Blood Coagul Fibrinolysis 2003a; 14:551-556.
Butenas S, van't Veer C and Mann KG. “Normal” thrombin generation. Blood 1999; 94:2169-2178.
Colucci M, Binetti B M, Branca M G, et al. Deficiency of thrombin activatable fibrinolysis inhibitor in cirrhosis is associated with increased plasma fibrinolysis. Hepatology 2003; 38:230-237.
Cvirn G, Gallisti S and Muntean W. Effects of antithrombin and protein C on thrombin generation in newborn and adult plasma. Thromb. Res. 1999; 93:183-190.
Cvirn G, Gallisti S, Leschnik B, et al. Low tissue factor pathway inhibitor (TFPI) together with low antithrombin allows sufficient thrombin generation in neonates. J Thromb Haemost 2003; 1:263-268.
Faber C G, Lodder J, Kessels F, et al. Thrombin generation in platelet-rich plasma as a tool for the detection of hypercoagulability in young stroke patients. Pathophysiol Haemost Thromb 2003; 33:52-58.
Giansily-Blaizot M, Al Dieri R, and Schved J-F. Thrombin generation measurement in factor VII-depleted plasmas compared to inherited factor VII-deficient plasmas. Pathophysiol Haemost Thromb 2003; 33:36-42.
He S, Antovic A, and Blombäck M. A simple and rapid laboratory method for determination of haemostasis potential in plasma. II. Modifications for use in routine laboratories and research work. Thromb Res 2001a; 103:355-361.
He S, Bremme K and Blombäck M. A laboratory method for determination of overall haemostatic potential in plasma. I. Method design and preliminary results. Thromb Res 1999; 96:145-156.
He S, Bremme K, Silveira A, et al. Hypercoagulation in surgical postmenopausal women having hormone replacement with overdose estradiol. Blood Coagul Fibrinolysis 2001b; 12:677-681.
Hemker HC and Béguin S. Phenotyping the clotting system. Thromb Haemost 2000; 84:747-50.
Hemker HC and Béguin S. Thrombin generation in plasma: its assessment via the endogenous thrombin potential. Thromb Haemost 1995; 74:134-138.
Hemker H C, Giesen P L A, Ramjee M, et al. The thrombogram: monitoring thrombin generation in platelet rich plasma. Thromb Haemost 2000; 83:589-591.
Laboratory measurements of hemostasis and thrombosis. In: Disorders of hemostasis and thrombosis: a clinical guide (Goodnight S H, Hathaway W E, eds), 2^{nd} ed. New York: McGraw-Hill, Inc. 2001; 20-30.
Lottermoser K, Petras S, Poge U, et al. The fibrinolytic system in chronic renal failure. Eur J Med Res 2001; 6:372-376.
Manco-Johnson M J, Nuss R, and Jacobson L. Heparin neutralization is essential for accurate measurement of factor VIII activity and inhibitor assays in blood samples drawn from implanted venous access devices. J Lab Clin Med 2000; 136:74-79.
Meh D A, Mosesson M W, DiOrio J P, et al. Disintegration and reorganization of fibrin networks during tissue-type plasminogen activator-induced clot lysis. Blood Coagul Fibrinolysis 2001; 12:627-637.
   Mosnier L O, Lisman T, van den Berg H M, et al. The defective down regulation of fibrinolysis in hemophilia A can be restored by increasing the TAFI plasma concentration. Thromb Haemost 2001; 86:1035-1039.

Palabrica T M, Liu A C, Aronovitz M J, et al. Antifibrinolytic activity of apolipoprotein(a) in vivo: human apolipoprotein(a) transgenic mice are resistant to tissue plasminogen activator-mediated thrombolysis. Nat Med 1995; 1:256-259.
Quiroga T, Goycoolea M, Giesen P L A, et al. Thrombin generation in platelet-poor plasma is normal in patients with hereditary mucocutaneous hemorrhages. Pathophysiol Haemost Thromb 2003; 33:30-35.
   Smith A A, Jacobson L J, Miller B I, et al. A new euglobulin clot lysis assay for global fibrinolysis. Thromb Res 2003; 112:329-337.
   Turecek P L, Varadi K, Keil B, et al. Factor VIII inhibitor-bypassing agents act by inducing thrombin generation and can be monitored by a thrombin generation assay. Pathophysiol Haemost Thromb 2003; 33:16-22.

TABLE 1a

| | CI | PT | aPTT | FVIII act | Fibrinogen |
|---|---|---|---|---|---|
| Group | (%) | (seconds) | (seconds) | (U/mL) | (mg/dL) |
| Newborn cord | 58 (43-77) | 13.8 (12.9-15.1) | 54.5 (48.7-61.9) | 86 (70-102) | 209 (186-233) |
| blood (n = 27) | | (n = 24) | (n = 24) | (n = 21) | |
| Normal children | 73 (53-95) | 12.3 (12.0-12.5) | 37.6 (34.7-39.3) | 117 (105-142) | 275 (237-320) |
| (n = 22) | | | | | |
| Normal adults | 115 (83-142) | 12.6 (12.3-13.1) | 34.8 (32.8-37.1) | 95 (86-105) | 292 (257-345) |
| (n = 22) | | | | | (n = 15) |
| Pregnant women | 239 (194-344) | 10.0 (10.0-10.4) | 33.0 (31.4-36.2) | 251 (212-288) | 484 (431-550) |
| (n = 24) | | (n = 22) | (n = 22) | (n = 17) | |

TABLE 1b

| | FI | PAI-1 Ag* | PAI-1 act* | ELT* |
|---|---|---|---|---|
| Group | (%) | (ng/mL) | (U/mL) | (minutes) |
| Normal cord blood | 210 (194-280) | 1.6 (1.2-2.6) | 3.9 (2.7-5.0) | 96 (48-120) |
| (n = 27) | | (n = 25) | (n = 25) | (n = 25) |
| Normal children | 140 (111-172) | 8.1 (3.8-13.4) | 17.4 (9.2-21.8) | 369 (258-423) |
| (n = 22) | | (n = 21) | (n = 21) | (n = 21) |
| Normal adults | 95 (88-119) | ** | ** | 354 (300-382) |
| (n = 22) | | | | (n = 9) |
| Pregnant women | 59 (50-74) | 20.4 (16.5-24.6) | 32.8 (31.1-39.0) | 507 (467-538) |
| (n = 24) | | | | |

Abbreviations:
CI = coagulation index;
FI = fibrinolytic index;
PT = prothrombin time;
aPTT = activated partial thromboplastin time;
sec = seconds;
FVIII = factor VIII;
act = activity;
PAI-1 = plasminogen activator inhibitor-1;
Ag = antigen;
ELT = euglobulin lysis time
*Published observed ranges [21] for PAI-1 Ag, PAI-act, and automated ELT in pregnant women, adults, children, and neonates (respectively) are as follows: PAI-1 Ag (ng/mL): 10.2-49.2, 0.5-27.5, 0.7-19.0, and 0.7-24.2; PAI-1 act (U/mL): 18.7-46.7, 1.9-28.4, 1.2-23.6, and 0.9-38.4; ELT (minutes): 393-690, 158-674, 159-654, and 21-387.
** Not assessed

TABLE 2
CloFAL assay CI values from individual coagulation
factor-deficient patient plasmas.

| Factor Deficiency* | CI |
|---|---|
| Fibrinogen | 0% |
| II | 13% |
| V | 0% |
| VII | 13% |
| VIII | 0% |
| IX | 3% |
| X | 4% |
| XI | 35% |
| XII | 52% |
| XIII | 61% |
| Prekallikrein | 119% |
| HMWK | 74% |

Abbreviations:
CI = coagulation index;
HMWK = high molecular weight kininogen
*The corresponding factor activity level of all factor-deficient plasmas was 1 U/dL in all cases, except factor II deficiency, where factor II activity was 3 U/dL. Fibrinogen concentration in fibrinogen-deficient plasma was 8 mg/dL.

TABLE 3

| | | | Factor | Factor |
|---|---|---|---|---|
| | Healthy | Healthy | VIII-deficient | VIII-deficient |
| | Adults | Children | Adults | Children |
| | (n = 25) | (n = 47) | (n = 18) | (n = 26) |
| Age (years)* | 35 | 5 (1-17) | 33 (18-79) | 8 (2-17) |
| | (21-53) | | | |
| Laboratory | | | | |
| designation^{†} | | | | |
| Moderate/Severe | — | — | 5 (28) | 7 (27) |
| Mild | — | — | 13 (72) | 19 (73) |
| Clinical | | | | |
| designation^{‡} | | | | |
| Moderate/Severe | — | — | 8 (47)^{§} | 5 (19) |
| Mild | — | — | 9 (53)^{§} | 21 (81) |

*Borderline statistical significance when comparing healthy vs. factor VIII-deficient children only (P = 0.05)
^{†}Severe factor VIII deficiency defined by activity of <1 U/mL by one-stage clotting assay
^{‡}Assessment of clinical severity according to personal bleeding history, using previously-published standardized criteria (see also Table 1)
^{§}Of n = 17 adults for whom clinical data on personal bleeding history was available

TABLE 4

| | Healthy | | Healthy | | Factor VIII-deficient | | Factor VIII-deficient | |
|---|---|---|---|---|---|---|---|---|
| | Adults | | Children | | Adults | | Children | |
| | (n = 25) | | (n = 47) | | (n = 18) | | (n = 27) | |
| CI (%)*^{†} | 94 | (43-162) | 71 | (21-225) | 1 | (0-51) | 5 | (0-49) |
| T₁(min)*^{†} | 25.5 | (20.3-33.0) | 33.4 | (14.3-58.5) | 48.8 | (30.0-90.8) | 67.5 | (37.5-177.8) |
| MA* | 0.384 | (0.246-0.532) | 0.335 | (0.186-0.650) | 0.316 | (0.017-0.486) | 0.324 | (0.033-0.646) |
| aPTT (sec)*^{†} | 37.1 | (30.7-42.0) | 39.1 | (30.0-43.8) | 53.2 | (41.4-111.4) | 54.7 | (40.2-121.4) |
| FVIII (U/dL)*^{†} | 102 | (66-196) | 141 | (92-228) | 16 | (0.4-49) | 11 | (0.2-42) |
| vWF Ag (%) | 97 | (66-144) | 94 | (52-164) | 94 | (42-307) | 98 | (45-202) |

Abbreviations:
CloFAL = Clot Formation and Lysis;
CI = coagulation index;
T₁ = time to maximal amplitude;
min = minutes;
MA = maximum amplitude;
aPTT = activated partial thromboplastin time;
sec = seconds;
FVIII = factor VIII activity (one-stage clotting assay);
vWF Ag = von Willebrand factor antigen
*Statistically significant when comparing healthy vs. factor VIII-deficient adults (P < 0.001 in all cases, except for MA, in which case P = 0.01)
^{†}Statistically significant when comparing healthy vs. factor VIII-deficient children (P < 0.001 in all cases)

TABLE 5

| | aPTT | CloFAL |
|---|---|---|
| Laboratory Designation | | |
| Moderate/severe (n = 12) | 100% | 100% |
| Mild (n = 32) | 88% | 94% |
| Clinical Designation^{†} | | |
| Moderate/severe (n = 11) | 100% | 100% |
| Mild (n = 32) | 88% | 94% |

Abbreviations:
CloFAL = Clot Formation and Lysis;
aPTT = activated partial thromboplastin time
^{†}Assessment of clinical severity according to personal bleeding history, as adapted from previously-published standardized criteria (reference 3; see also Table 1)

1. A global hemostatic assay method comprising:
obtaining a sample; and
measuring both clot formation and fibrinolysis in the sample.

2. The method of claim 1, wherein clot formation and fibrinolysis are measured simultaneously.

3. The method of claim 1, wherein the sample comprises a platelet-poor plasma sample.

4. The method of claim 1, wherein the sample comprises a pre-operative screening test sample.

5. The method of claim 1, wherein clot formation and fibrinolysis are measured by optical density.

6. The method of claim 5, wherein optical density is determined using a spectrophotometer.

7. A global hemostatic assay method comprising:
obtaining a sample;
adding a buffered reactant solution to the sample, wherein the solution contains at least one activator of coagulation and at least one activator of clot lysis; and
measuring both clot formation and fibrinolysis in the sample.

8. The method of claim 7, wherein clot formation and fibrinolysis are measured simultaneously.

9. The method of claim 8, wherein clot formation and fibrinolysis are measured by optical density.

10. The method of claim 9, wherein optical density is determined using a spectrophotometer.

11. The method of claim 7, wherein clot formation and fibrinolysis are measured continuously for a period from 1 to 3 hours.

12. The method of claim 11, wherein clot formation and fibrinolysis are measured continuously for a period from 2 to 3 hours.

13. The method of claim 11, wherein clot formation and fibrinolysis are measured continuously for a period from 1 to 2 hours.

14. The method of claim 7, wherein clot formation and fibrinolysis are measured at frequent time intervals for a period from 1 to 3 hours.

15. The method of claim 14, wherein the time interval is selected from the group consisting of 5, 10, 15, 20, 25, 30, 35, 40, 45, 50, 55, and 60 seconds.

16. The method of claim 7, wherein the activator of coagulation is selected from calcium, tissue factor (TF), thrombin, phospholipid reagent or a combination thereof.

17. The method of claim 7, wherein the activator of fibrinolysis is selected from tissue-type plasminogen activator (tPA), urokinase-type plasminogen activator (uPA, or urokinase), plasmin, a carboxypeptidase, potato tuber carboxypeptidase inhibitor or a combination thereof.

18. The method of claim 7, wherein the sample is obtained from a subject selected from the group consisting of a human, a dog, a cat, a horse, a cow, a sheep, a goat and a non-human mammal.

19. The method of claim 18, wherein the subject has or is suspected of having a heart condition.

20. The method of claim 18, wherein the subject has or is suspected of having an abnormal blood condition.

21. The method of claim 20, wherein the abnormal blood condition is selected from the group consisting of von Willebrand's disease, severe hemophilia A, severe hemophilia B, other coagulation factor deficiency, other coagulation factor dysfunction, afibrinogenemia, hypofibrinogenemia, dysfibrinogenemia, hepatic dysfunction, cirrhosis, renal dysfunction and a combination thereof.

22. The method of claim 20, wherein the abnormal blood condition is selected from the group consisting of factor V Leiden mutation, prothrombin 20210 mutation, methylene tetrahydrofolate reductase (MTHFR) mutation deficiency, protein C deficiency, protein S deficiency, antithrombin deficiency, other native anticoagulant deficiencies, activated protein C resistance, coagulation factor excess, excess of factor IIa, excess of factor VII, excess of factor VIII, excess of factor IX, excess of factor XI, antiphospholipid antibodies, lupus anticoagulant, anticardiolipin antibodies, beta-2 glycoprotein-1, elevated plasma homocysteine, elevated serum homocysteine, elevated plasma lipoproteins, elevated serum lipoproteins, elevated lipoprotein[a], dyslipidemia, hypercholesterolemia and a combination thereof.

23. The method of claim 19, further comprising comparing coagulation and fibrinolysis in a sample from a normal subject and a sample from a subject with a disease or heart condition.

24. A kit for analyzing a plasma sample comprising:
a buffered reactant solution;
an activator of coagulation; and
an activator of fibrinolysis.

25. A global assay method comprising:
obtaining a plasma sample from a subject;
assessing at least two parameters of the plasma sample;
calculating the clotting index and the fibrinolysis index from the parameters; and
treating the subject with at least one therapeutic agent.

26. The method of claim 25, wherein the parameters are selected from the group consisting of maximum amplitude of spectrophotometric absorbance, time to maximum turbidity, time to completion of the first phase of decline in turbidity, area under the curve of spectrophotometric absorbance over a measured time interval and time from assay initiation to clot initiation as measured by optical density over a baseline threshold value and a combination thereof.

27. The method of claim 25, further comprising obtaining a plasma sample before, during and after treating the subject with at least one therapeutic agent.

28. The method of claim 1, wherein the sample comprises a platelet-rich plasma sample.

29. The method of claim 19, wherein activation of coagulation is inhibited by corn trypsin inhibitor or other contact activation inhibitor.

30. The method of claim 19, further comprising adding white blood cells obtained from the subject or obtained from a standard source.

31. The method of claim 19, further comprising adding endothelial cells obtained from the subject or obtained from a standard source.
